(19) United States
(12) Patent Application Publication
(10) Pub. No.: US 2011/0257273 A1
(43) Pub. Date: Oct. 20, 2011

(54) POLYESTER RESIN AND PURPOSES THEREOF

(75) Inventors: Naoya YABUUCHI, Osaka (JP); Hirofumi YAMASHITA, Osaka (JP); Koji MORITA, Osaka (JP); Yasunori MIWA, Osaka (JP)

(73) Assignee: NIPPON BEE CHEMICAL CO., LTD., Hirakata-City (JP)

(21) Appl. No.: 13/084,127

(22) Filed: Apr. 11, 2011

(30) Foreign Application Priority Data
Apr. 20, 2010 (JP) 2010-096916
Aug. 23, 2010 (JP) 2010-185963

(51) Int. Cl. A61K 8/85; C09D 167/02; C07C 69/54; C09D 133/08; C08G 63/91; C09D 11/10; C08G 63/16; C08G 18/00

(52) U.S. Cl. 514/772.3; 528/272; 528/302; 524/604; 524/513; 521/172; 524/560; 525/440.01; 524/589; 525/440.07; 560/205

(57) ABSTRACT

A low-molecular-weight polyester resin which can elasticize a resin suitably and can be used for various purposes, various resins obtained by using the resin, and the purposes thereof. The polyester resin is obtained by polymerizing a monomer composition containing 10 to 90 weight % of a linear dicarboxylic acid and/or diol having at least 8 carbon atoms (I), 5 to 80 weight % of a branched dicarboxylic acid and/or diol having at least 4 carbon atoms (II-1) and/or 2 to 40 weight % of at least one polyfunctional monomer (II-2) selected from the group consisting of polyols, polycarboxylic acids and hydroxycarboxylic acids having 3 or more functional groups respectively and which has the number average molecular weight of 500 to 5000 and is amorphous.

### TECHNICAL FIELD

The present disclosure relates to a polyester resin, and a coating composition, an adhesive composition, a polyurethane foam, resin particles, cosmetics, a matte coating composition, an acrylic monomer, an energy curable coating composition, curable resin composition, a hot-melt adhesive composition, a print ink composition, an energy curable resin, and an energy curable adhesive composition, which obtained by using the polyester resin.

### BACKGROUND OF THE DISCLOSURE

For coating compositions, resin particles, energy curable coating compositions, inks, adhesives, energy curable adhesives, and urethane foams, it is sometimes required to add the elasticity to resins to be used. For example, in the case of the coating compositions for automobiles, the elasticity is added to obtain the scratch resistance. In the case of resin particles, the elasticity is added to improve the texture when contained in coating compositions and cosmetics. In the case of the energy curable coating compositions, it is desired to add the impact resistance property by adding the elasticity to a coating film. A polyol is used for various purposes such as adhesives, hot-melt adhesives, print inks, and energy curable adhesives, and it is required to improve the performance of the polyol.

As such components to add elasticity, methods using aliphatic polyester resins such as polycaprolactone, or acrylic resins of which a fatty acid chain is combined at the side chain are disclosed in Patent document 1. However, such the coating composition cannot obtain sufficient effects because a good balance between the scratch resistance and another properties cannot be kept, for example, sometimes the scratch resistance is good but the water resistance and the humidity resistance are insufficient, and vice versa.

In patent document 2, a biodegradable polyester polyurethane solution obtained by using an aliphatic polyester resin is disclosed. However, Patent document 2 discloses only a polyurethane solution which is obtained by reacting a polyester resin having the weight average molecular weight of 10000 or more with a polyisocyanate compound, and the solution is not preferred as a material for coating compositions from the viewpoint of the solubility, the compatibility with another compounds, and the crystallinity. In particularly, because of high crystallinity, there is a problem that defects of coating workability and adhering workability tend to take place.

In patent documents 3 and 4, a polyester obtained by using sebacic acid which can be used for various purposes is disclosed. However, there is a problem that defects of workability and properties of the cured material tend to take place when the polyester is used for various purposes, because the polyester resin obtained by using sebacic acid as an acid component has high crystallinity.

On the other hand, as a coating composition material, a cosmetic material, and other industrial materials, resin particles made with resins are produced. Concerning such resin particles, it is required to produce resin particles having higher elasticity.

Further, various kinds of acrylic ester compounds are used as an energy curable resin component. Such the energy curable resin is disclosed in, for example, Patent document 5. Patent document 5 does not disclose that the impact resistance of the coating film can be improved by increasing the elasticity of the coating film formed by using the energy curable coating composition.

### PRIOR TECHNICAL DOCUMENT

### Patent Document

### [Patent Document 1] Japanese Kokai Publication 2003-253191

### [Patent Document 2] Japanese Kokai Publication 2006-233119

### [Patent Document 3] Japanese Kokai Publication 2010-84109

### [Patent Document 4] Japanese Kokai Publication Hei03-239715

### [Patent Document 5] Japanese Kokai Publication 2009-221457

### NONPATENT DOCUMENT

[Nonpatent Document 1] Society of Polymer Science, Japan, natural material plastic, first edition, KYORITSU SHUPPAN CO., LTD, 2006

### SUMMARY OF INVENTION

### Problem to be Solved by the Invention

The object of the present disclosure which has been in view of the above-mentioned state of the art, is to provide a low-molecular-weight polyester resin which can elasticize a resin suitably and can be used for various purposes, various resins obtained by using the resin, and the purposes thereof.

### Means for Solving Object

The present disclosure relates to a polyester resin obtained by polymerizing a monomer composition containing 10 to 90 weight % of a linear dicarboxylic acid and/or diol having at least 8 carbon atoms (I), 5 to 80 weight % of a branched dicarboxylic acid and/or diol having at least 4 carbon atoms (II-1) and/or 2 to 40 weight % of at least one polyfunctional monomer (II-2) selected from the group consisting of polyols, polycarboxylic acids and hydroxycarboxylic acids having 3 or more functional groups respectively and which has the number average molecular weight of 500 to 5000 and is amorphous.

Preferably, part or all of the linear dicarboxylic acid and/or diol having at least 8 carbon atoms (I) is sebacic acid.

The polyester resin is preferably obtained by polymerizing a monomer composition containing 0 to 88 weight % of other monomer (III).

The other monomer (III) preferably comprises at least one monomer selected from the group consisting of succinic acid, polyethylene glycol, 1,3-propanediol, and 1,4-butanediol.

The present disclosure relates to a polyester resin which is obtained by polymerizing a monomer composition containing a dicarboxylic acid monomer comprising at least one dicarboxylic acid (a) selected from the group of succinic acid and sebacic acid, a diol monomer comprising 1,4-butanediol and/or 1,3-propanediol (b), and at least one polyfunctional monomer (c) selected from the group of polyols, polycarboxylic acids and hydroxycarboxylic acids having 3 or more functional groups respectively, wherein said monomer composition comprises 40 to 95 weight % of bio-based materials relative to the all resin materials, the number average molecular weight (Mn) thereof is 500 to 5000, and said polyester resin is amorphous.

The present disclosure relates to a coating composition containing a polyester resin (A) and a curing agent (B), wherein the polyester resin (A) is the above-mentioned polyester resin.

The coating composition preferably comprises a hydroxyl group-containing acrylic resin (C).

The present disclosure relates to an adhesive composition containing a polyester resin (A) and a curing agent, wherein the polyester resin (A) is the above-mentioned polyester resin.

The present disclosure relates to a polyurethane foam obtained by foaming a composition containing the above-mentioned polyester resin (A) and a polyisocyanate (B-1).

The present disclosure relates to a resin particle obtained by suspension polymerization of the coating composition, and having the number average particle diameter of 2 to 20 μm.

The present disclosure relates to a cosmetic containing the resin particle.

The present disclosure relates to a matte coating composition containing the resin particle.

The matte coating composition is preferably a water-borne coating composition.

The present disclosure relates to an acrylic monomer obtained by converting an end of the polyester resin to an acryloyl group.

The present disclosure relates to an energy curable coating composition of which part or all is the acrylic monomer.

The present disclosure relates to a curable resin composition having a terminal isocyanate group obtained by reacting the above-mentioned polyester resin (A) and a plyisocyanate (B-1).

The present disclosure relates to a moisture-curable reactive hot-melt adhesive composition containing the curable resin composition.

The present disclosure relates to a resin composition having a terminal hydroxyl group obtained by reacting the above-mentioned polyester resin (A) and a polyisocyanate (B-1).

The present disclosure relates to a print ink composition containing the resin composition having a terminal hydroxyl group.

The present disclosure relates to an energy curable resin by reacting the polyester resin (A), a compound having an unsaturated group and a functional group which is reactive with an isocyanate group, and a polyisocyanate (B-1).

The present disclosure relates to an energy curable adhesive composition containing the energy curable resin.

### EFFECT OF THE INVENTION

The present disclosure produces a polyester resin which can provide good elasticity with a resin, a coating composition, resin particles, cosmetics, a matte coating composition, an acrylic monomer, and an energy curable coating composition containing the polyester resin. These products can be obtained by using bio-based materials as the above-mentioned resin.

### BRIEF DESCRIPTION OF THE DRAWINGS

FIG. 1 is a figure of showing the way of attaching on evaluation process of adhesive compositions obtained in examples.

### DETAILED DESCRIPTION OF PREFERRED EMBODIMENTS

### (Polyester Resin)

The first polyester resin of the present disclosure is a polyester resin which contains a linear structure having 8 or more carbon atoms in a molecule and is amorphous. Because of such structure, it can provide the elasticity with a coating film, resin particles and so on. Furthermore, because the polyester resin is amorphous, it is superior in the miscibility with other components and suitable for applications in conjunction with other components.

The polyester resin of the present disclosure is preferred because it can be obtained by using bio-based materials at a high rate as raw materials.

Currently, plastics are used in various fields of life and industrials; the amount of production has been remarkably increased. Most of such plastics are obtained by chemical synthesis of mineral materials such as petroleum and natural gas. Incineration treatment of the waste derived from the plastics results in carbon dioxide emissions. This carbon dioxide contributes to global warming.

There is the same problem with a plastic-derived coating agent as the above-mentioned problem, because a coated article, which is no longer required after used, is generally incineration treated or disposed in the ground after separating the coating film from the substrate.

Therefore, a plastic made of natural material is proposed as a substitute for a traditional plastic made from fossil resource which causes the above-mentioned problem, for example, bio-based polymers such as polyhydroxyalkanoic acid, polyalkylene succinate, and polysaccharides are proposed. (See nonpatent document 1).

These bio-based polymers have been discussed in hope of it's biodegradability and the biodegradable capability by microorganisms in the ground has been required. In recent years, the study to reduce the discharge amount of carbon dioxide has begun to be taken quite seriously. So, the study of bio-based polymers in different way form biodegradability became necessary. Additionally, it is desired to use the bio-based polymer components as resin particles used in coating compositions and cosmetics, and materials for energy curable coating compositions. The first polyester resin of the present disclosure is which can accomplish the task of the reduction of the discharge amount of carbon dioxide, because it contains the components obtained from bio-based materials as the main constituent unit in the chemical constitution thereof so that it can contain a high percentage of bio-based materials.

The first polyester resin of the present disclosure contains 10 to 90 weight % of a linear dicarboxylic acid and/or diol having at least 8 carbon atoms (I). The added amount proportion in the present disclosure is calculated according to the added amount proportion of carboxylic acid, polyols, and hydroxycarboxylic acid to be used as raw materials.

As the linear dicarboxylic acid having at least 8 carbon atoms, there may be mentioned, for example, suberic acid, azelaic acid, sebacic acid, undecanedioic acid, dodecanedioic acid, and so on. As the linear diol having at least 8 carbon atoms, there may be mentioned, for example, 1,8-octanediol, 1,9-nonanediol, 1,10-decanediol, and so on. The upper limit of carbon atoms of the linear dicarboxylic acid and/or diol having at least 8 carbon atoms (I) is not particularly limited but 18 is preferred.

When the linear dicarboxylic acid and/or diol having at least 8 carbon atoms (I) is less than 10 weight %, it is not preferred because the scratch resistance, the water resistance, the humidity resistance, and the weather resistance are insufficient. In addition, when the linear dicarboxylic acid and/or diol having at least 8 carbon atoms (I) is more than 90 weight %, there is a problem that the crystallinity of the polyester resin becomes higher to degrade the miscibility with other resins. The upper limit of the added amount of the linear dicarboxylic acid and/or diol having at least 8 carbon atoms (I) is preferably 70 weight %, and the lower limit is preferably 20 weight %.

Part or all of the linear dicarboxylic acid and/or diol (I) having at least 8 carbon atoms is preferably sebacic acid. Sebacic acid is particularly preferred because bio-based one is readily-accessible and the resin obtained by using sebacic acid is superior in the properties including the scratch resistance, the water resistance, the humidity resistance, the weather resistance, and hardness.

The primary polyester resin of the present disclosure contains 5 to 80 weight % of a branched dicarboxylic acid and/or diol (II-1) having at least 4 carbon atoms and/or 2 to 40 weight % of at least one polyfunctional monomer (II-2) selected from the group consisting of polyols having, polycarboxylic acids and hydroxycarboxylic acids having 3 or more functional groups respectively. That is to say, the polyester resin contains a branched dicarboxylic acid having at least 4 carbon atoms, a branched diol having at least 4 carbon atoms (1-1), and/or at least one polyfunctional monomer (II-2) selected from the group consisting of polyols, polycarboxylic acids and hydroxycarboxylic acids having 3 or more functional groups respectively in definite proportion.

Compounds corresponding to the above-mentioned (II-1) and (II-2) can decrease the crystallinity degree of the polyester resin to obtain amorphous polyester resin when it is used with the monomer (I). When the compound (II-1) is used as such the copolymerization component, the added amount is needed to be within the range of 5 to 80 weight %. When the component (II-2) is used, the added amount is needed to be within the range of 2 to 40 weight %.

The branched dicarboxylic acid having at least 4 carbon atoms is not particularly limited but includes, for example, methylsuccinic acid, dimethylsuccinic acid, ethylsuccinic acid, 2-methylglutaric acid, 2-ethylglutaric acid, 3-methylglutaric acid, 3-ethylglutaric acid, 2-methyladipic acid, 2-ethyladipic acid, 3-methyladipic acid, 3-ethyladipic acid, and methylglutaric acid.

The branched diol having at least 4 carbon atoms is not particularly limited but includes 1,2-butanediol, 1,3-butanediol, 1,2-pentanediol, 1,3-pentanediol, 1,4-pentanediol, 2,3-pentanediol, 2,4-pentanediol, 1,2-hexanediol, 1,3-hexanediol, 1,4-hexanediol, 1,5-hexanediol, 3-methyl-1,5-pentanediol, and neopentyl glycol.

The upper limit of the carbon atoms of the component (II-1) is not particularly limited but preferably 8.

When the added amount of the component (II-1) is less than 5 weight % it becomes difficult to make the polyester resin amorphous. When the added amount of the component (II-1) is more than 80 weight %, insufficient elasticity can not be obtained because the added amount of the component (I) is low excessively. The lower limit of the component (II-1) to be added is preferably 10 weight %, and the upper limit is preferably 40 weight %.

As the polyfunctional monomer (II-2) selected from the group consisting of polyols, polycarboxylic acids and hydroxycarboxylic acids having 3 or more functional groups respectively, there may be mentioned polycarboxylic acids such as methylcyclohexene tricarboxylic acid, and trimellitic acid; polyols having 3 or more functional groups such as trimethylolpropane, pentaerythritol, glycerin, mannitol, and xylitol, and hydroxycarboxylic acids such as 2,5-dihydroxybenzoic acid. So, the crystallinity of the resin is decreased to make the resin amorphous by using the component, and a resin which is suitable as a material of a coating composition, an adhesive, and urethane foam can be obtained.

The amount of the component (II-2) is 2 to 40 weight %. If less than 2 weight %, it is hard to degrade the crystallinity degree to a satisfactory extent. If more than 40 weight %, it becomes impossible to achieve sufficient elasticity because the crosslinking density becomes too high. The upper limit of the added amount of the component (II-2) is preferably 20 weight %, and the lower limit is preferably 3 weight %.

The first polyester resin of the present disclosure may contain two or more compounds belonging to the components (II-1) and (II-2).

The first polyester resin of the present disclosure has the number average molecular weight (Mw) of 500 to 5000. The resin is comparatively low-molecular-weight polyester resin to be obtained by solution polymerization and so on. The solubility of the resin to a solvent can be improved and the use as a coating composition becomes easier by making the resin low-molecular-weight. Furthermore, there is the advantage that the miscibility with other components can be improved on reaction even if the resin is reacted with other components. There is the advantage that it can prevent a compound obtained by reacting the resin with other compounds from being high viscosity.

The number average molecular weight of the polyester resin of the present disclosure is calculated in terms of polystyrene by using GPC. More specifically, the number average molecular weight is calculated by using HLC-8220 GPC manufactured by Tosoh Corporation, and TSK gel Super Multipore HZ-M as column.

The first polyester resin of the present disclosure is amorphous. The miscibility with other components can be improved and the resin can be added to a coating composition easily, because the resin is amorphous. In the present disclosure, “amorphous” means that the crystal melting heat is 0 to 5 cal/g measured by DSC method (JIS K 7121). The crystal melting heat is preferably 0 to 3 cal/g.

The measurement of the crystal melting heat is done according to the following method, more specifically. A resin 5 to 10 mg obtained by removing the solvent is put in an aluminum pan and mounted on a differential scanning calorimeter (DSC-2 manufactured by PerkinElmer Co., Ltd.). After heating to 200° C. at the rate of temperature increase of 10° C./min, the resin is cooled to 25° C. and heated at the rate of temperature increase of 10° C./min. Then, the crystal melting heat is calculated whole crystal peak area of DSC chart.

The polyester resin of the present disclosure may comprise 0 to 88 weight % of polyols, polycarboxylic acids, and hydroxycarboxylic acids other than the components (I), (II-1), and (II-2) (hereinafter referred to as the other monomer (III)). The various monomers can be polymerized according to use, but it is not preferred to add the above-mentioned component (III) more than 88 weight % because the property that can give the elasticity and can achieve high compatibility with other resins may be decreased.

The other monomer (III) is not particularly limited, but includes, for example, aromatic dicarboxylic acids such as phthalic acid, 2,6-naphthalenedicarboxylic acid and 2,7-naphthalenedicarboxylic acid, and anhydrides thereof; saturated aliphatic dicarboxylic acids such as succinic acid, adipic acid, and 1,4-cyclohexanedicarboxylic acid; diols such as 1,3-propanediol, 1,4-butanediol, 1,5-pentanediol, diethylene glycol, triethylene glycol, 1,4-cyclohexanediol, 1,4-cyclohexane dimethanol, bisphenol A alkyleneoxide adducts, bisphenol S alkylene oxide adducts, and 1,2-propanediol; lactones such as γ-butyrolactone, and ε-caprolactone and hydroxycarboxylic acids corresponding to these compounds; aromatic oxymonocarboxylic acids such as p-oxyethoxybenzoic acid. Among them, succinic acid, polyethylene glycol, 1,3-propanediol, and 1,4-butanediol are preferred.

In the polyester resin of the present disclosure, it is not preferred to use ethylene glycol as the other monomer (III). When ethylene glycol is used, it may make it difficult to reduce the crystallinity sufficiently. Further, ethylene glycol is low-molecular weight, so the ester bond number per unit weight is large and the properties such as the hydrolysis resistance may get worse. More specifically, the proportion of ethylene glycol is preferably less than 5 weight % relative to the total raw materials

As raw materials for the components (I), (II-1), (II-2), and (III), it is preferred to use bio-based materials. This is to respond to the reduction of CO₂. More preferably, it is preferred to use bio-based materials at a rate of 40 to 95 weight % relative to the all raw materials.

The second polyester resin of the present disclosure contains a dicarboxylic acid (a), a diol (b), and a polyfunctional monomer (c) as essential components, wherein the dicarboxykic acid-derived structure has succinic acid and/or sebacic acid unit, the diol derived-structure contains 1,4-butanediol and/or 1,3-propanediol, contains 40 to 95 weight % of bio-based materials relative to the all raw materials, and is amorphous polyester resin.

It is preferred to obtain the polyester resin having such the composition because the bio-based components can be contained in the resin at a high rate. The resin having such the composition is amorphous, so the resin is superior in compatibility with other components when added in a coating composition and so on. Therefore, the resin can be used for the same use as that of the primary polyester resin of the present disclosure.

As for succinic acid, 1,4-butanediol, and 1,3-propanediol being the monomer components as the raw materials of the second polyester resin of the present disclosure, many compounds made of petroleum feedstock are marketed, industrially. On the other hand, bio-based synthesis methods of these compounds are established, so these are available compounds as bio-based materials. In the present disclosure, it is important to use a monomer which can be synthesized from bio-based materials easily as a major ingredient, because the polyester resin contains bio-based materials of 40 to 95 weight % relative to the all raw materials.

In the present disclosure, succinic acid, sebacic acid, 1,4-butanediol, and 1,3-propanediol that are made of petroleum feedstock may be used together as raw materials, as long as the polyester resin contains bio-based raw materials of 40 to 95 weight % relative to the all resin materials. More preferably, the resin contains succinic acid, sebacic acid, 1,4-butanediol, and 1,3-propanediol that are made of bio-based materials of 40 to 95 weight % relative to the all resin materials.

In the second aspect of the present disclosure, bio-based materials other than the above-mentioned materials may be used together. For example, there may be mentioned glycerin, lactic acid, adipic acid, and 3-hydroxybutanoic acid as other bio-based materials.

The second polyester resin of the present disclosure is preferably obtained by using succinic acid, sebacic acid, 1,4-butanediol, and 1,3-propanediol at a rate of 40 to 95 weight % relative to the all materials and using monomers made of petroleum feedstock at a rate of 60 to 5 weight % relative to the all materials, not using the other bio-based materials.

The second polyester resin of the present disclosure contributes to the reduction of carbon dioxide because the resin contains the bio-based materials of 40 weight % or more relative to the all materials weight. The added amount is needed to be 95 weight % or less because it is difficult to use bio-based materials of 100 weight % for suitable property as a material of a coating composition, an adhesive, and urethane foam.

A resin produced from a monomer composition containing a lot of bio-based materials is treated as a resin which releases small amount of carbon dioxide into the environment on such treatments as incineration process. So, in recent years that the regulation on the discharge amount of carbon dioxide is strengthened, the resin can be used as a resin with least adverse impact on environment.

The second polyester resin of the present disclosure obtained by using at least one polyfunctional monomer (c) selected from the group consisting of polyols, polycarboxylic acid, and hydroxycarboxylic acid having 3 or more functional groups respectively. By using these compounds, the crystallinity of the resin is decreased to make the resin amorphous and suitable for a coating composition.

As the polyfunctional monomer (c) which can be used in the second polyester resin of the present disclosure, there may be mentioned, for example, polycarboxylic acids such as trimellitic acid; polyols having 3 or more functional groups such as trimethylolpropane, glycerin, mannitol, and xylitol; and so on. Bio-based materials may be used as the polyfunctional monomer (c), and other nonbio-based materials such as petroleum-based materials may be used. Two or more kinds of these materials may be used.

The polyol, polycarboxylic acid, and hydroxycarboxylic acid is preferably contained at a rate of 5 to 25 weight % in terms of structure unit in a resin. By containing in the above-mentioned range, it is possible to make the obtained resin amorphous, which has a suitable property for coating composition.

The second polyester resin of the present disclosure can be obtained by using monomers not being bio-based component in the range of 60 to 5 weight % as raw materials. The monomer not being bio-based component includes monomers being petroleum-based components. There may be mentioned trimethylolpropane, and pentaerythritol as petroleum-based polyols having 3 or more functional groups, and petroleum-based components such as trimellitic acid, and pyromellitic acid as polycarboxylic acids having 3 or more functional groups.

As the monomer having two functional groups being a petroleum-based component, there may be mentioned polycarboxylic acid components such as 1,4-cyclohexanedicarboxylic acid, 3-methyl-1,5-pentanediol, 1,2-cyclohexanedicarboxylic acid, terephthalic acid, isophthalic acid, azelaic acid, and maleic acid; diol components such as 1,6-hexanediol, neopentyl glycol, 1,9-nonane diol, and hydrogenated bisphenol A; lactones such as ε-caprolactone.

Among them, 1,2-cyclohexanedicarboxylic acid, 3-methyl-1,5-pentanediol, and neopentyl glycol are preferred. In the present disclosure, acid anhydrides can be used arbitrarily. These monomers are preferred because it becomes easy to decrease the crystallinity of the resin and make the resin amorphous. Preferably, these monomers are contained in the range of 10 to 40 weight % relative to all the raw materials.

In addition, in the raw materials of the second polyester resin of the present disclosure, the content rate of hydroxycarboxylic acid is 10 weight % or less, preferably 5 weight % or less with respect to the hydrolysis resistance.

The second polyester resin of the present disclosure is amorphous. The miscibility of the resin with other components can be increased and the resin can be added easily into a coating composition easily because the resin is amorphous. In addition, in the second aspect of the present disclosure, “amorphous” means the same definition mentioned in the first aspect of the present disclosure.

It is preferred to adjust the composition of resin materials to obtain such amorphous polyester resin. Generally, it becomes easy to make the resin amorphous by combining various materials, because the crystallinity degree increases when the same aliphatic dicarboxylic acid and a single diol are used together.

The polyester resin of which crystal melting heat is in the above-mentioned range is particularly preferred in terms of such superior properties as transparency, compatibility with a curing agent, pigment dispersant ability, and coating workabikity.

The second polyester resin of the present disclosure has the number average molecular weight (Mn) of 500 to 5000. That is, it is a polyester resin which is a comparatively-low molecular weight polyester resin to an extent that it can be obtained by solution polymerization and so on. Because the resin is low molecular weight, the solubility to a solvent increases and the use as a coating composition becomes easier. In addition, in the case of reacting with other components, there is the advantage that the miscibility with other components on reaction becomes better. There is the advantage that the viscosity of compound obtained by reacting with other compounds is prevented from increasing excessively. The number average molecular weight (Mn) is more preferably 600 to 4000.

The number average molecular weight of the polyester resin of the present disclosure is measured by GPC in terms of polystyrene. The number average molecular weight is, more specifically, measured by using HLC-8220 GPC manufactured by Tosoh Corporation, and TSK gel Super Multipore HZ-M as column

The polyester resin of the present disclosure, including the first polyester resin and the second polyester resin, can be obtained by common production methods of polyester resins. More specifically, for example, the resin can be obtained by a method comprising mixing the above-mentioned materials and dehydrating to polycondensate. The dehydration to polycondensate can be conducted by using solvents that are azeotropic for water such as toluene, and xylene under ordinary pressure at 150 to 240° C. In addition, the polymerization can be conducted under reduced pressure of about 1 to 20 mmHg, without using the solvents.

In the polymerization of the polyester resin, polymerization catalysts such as tin oxide and dibutyltin dilaurate may be used.

The polyester resin of the present disclosure, including the first polyester resin and the second polyester resin, has preferably the hydroxyl group value of 60 to 260, more preferably 70 to 220. The lower limit of the hydroxyl group value is more preferably 120. When the hydroxyl group value is less than 60, the crosslinking density of a coating film to be obtained may be decreased, and when over 260, the adhesion may be degraded.

The polyester resin of the present disclosure, including the first polyester resin and the second polyester resin, if the resin is used in the form of water dispersant, preferably has the acid value of 4 to 120 mgKOH/g. More preferably, the acid value is 10 to 60 mgKOH/g. When the acid number is less than 4 mgKOH/g, the dispersing stability in water of the polyester resin may be decreased. When more than 120 mgKOH/g, the water resistance of a coating film to be obtained may be degraded.

The polyester resin of the present disclosure, including the first polyester resin and the second polyester resin, preferably has the glass transition temperature (Tg) of −40 to 80° C., more preferably −20 to 40° C. When the glass transition temperature is less than the lower limit, the hardness of the resin may be decreased. When more than the upper limit, the obtained resin may be hard and fragile.

The polyester resin of the present disclosure, including the primary polyester resin and second polyester resin, can be used as a resin component in a coating composition, a material for a resin particle, a material for an energy curable resin, a resin component for an adhesive, a material for various curable resin composition, and so on. Specifically, the resin can be used as a soft segment because the resin contains linear aliphatic structure units at a high rate. Therefore, it can be used as a material or a constitution unit of a material for a coating composition requiring the scratch resistance, or resin particles requiring the elasticity. The polyester resin of the present disclosure may be used in the various forms such as a resin solution, a resin dispersion, and a solid. When the polyester resin is used for these purposes, the polyester resin of the present disclosure is preferably used in the resin solution form obtained by dissolving the resin in an organic solvent, or in the resin dispersion form obtained by dispersing the resin in water.

The purposes of the polyester resin of the present disclosure is discussed in more detail below

### (Coating Composition)

The first and the second polyester resin of the present disclosure can be used as resin binders in coating compositions. More specifically, in a coating composition comprising a polyester resin (A), a curing agent (B), and a hydroxyl group-containing acrylic resin (C) which is used according to need, the polyester resins can be used as the polyester resin (A) component.

The coating composition comprising a polyester resin not only contributes to environmental conservation but also has good durability and appearance so that it can be used suitably for coating of automobiles, home electronics, and so on. A coating film having good scratch resistance can be obtained by using it because the resin can form a coating film having the elasticity. Such the coating composition can be used as a clear coating composition that forms the top coat in the automobile coating.

The curing agent (B) is not particularly restricted but may include compounds containing 2 or more functional groups which can reacts with a hydroxyl group, carboxyl group, and so on. These compounds include, for example, polyisocyanate compounds; amino resins such as melamine resin.

The polyisocyanate is not particularly restricted as long as the polyisocyanate is a compound containing 2 or more isocyanate groups but includes, for example, aromatic compounds such as tolylenediisocyanate, 4,4′-diphenylmethane diisocyanate, xylylene diisocyanate, and metaxylylene diisocyanate; aliphatic compounds such as hexamethylene diisocyanate; alicyclic compounds such as isophorone diisocyanate; monomers thereof and polymer types such as burette type, nurate type, and adducts type.

The marketed products of the polyisocyanate includes Duranate 24A-90PX (NCO:23.6%, product name, manufactured by Asahi Kasei Chemicals Corporation), Sumidur N-3200-90M (product name, manufactured by Sumitomo Bayer Urethane Co., Ltd.), TAKENATE D165N-902X (product name, manufactured by Mitsui Takeda Chemicals), Sumidur N-3300, Sumidur N-3500 (product name, manufactured by Sumitomo Bayer Urethane Co., Ltd.), and Duranate THA-100 (product name, manufactured by Asahi Kasei Chemicals Corporation). According to need, blocked isocyanates that obtained by blocking these compounds can be used.

An equivalent ratio between isocyanate groups in said curing agent (B) and sum of hydroxyl groups in said polyester resin (A) and hydroxyl groups in said hydroxyl group-containing acrylic resin (C) (NCO/OH) is preferably 0.8/1 to 1.2/1. If less than 0.8/1, the obtained clear coating film may be insufficient in coating film strength. If over 1.2/1, the weather resistance and the hardness may become insufficient. The equivalent ratio (NCO/OH) is more preferably 0.9/1 to 1.1/1.

The amino resin is a condensed product obtained by modifying a condensed product of an amino compound such as melamine, urea, and benzoguanamine with an aldehyde compound such as formaldehyde and acetaldehyde by a lower alcohol such as methanol, ethanol, propanol, and butanol.

The amino resin preferably has the molecular weight of 500 to 2000. As such amino resin, there may be mentioned melamine resins that sold, called trademark Cymel 235, 238, 285, and 232 (manufactured by Mitsui Cytech, Ltd.).

The amount to be added of the melamine resin preferably has 15 weight parts of the lower limit and 35 weight parts of the upper limit relative to 100 weight parts of solid matter in the coating composition. If the amount is less than 15 weight parts, properties such as the curability may be decreased. If the amount is over 35 weight parts, the adhesion property and the heated water resistance may be descended. The lower limit is more preferably 20 weight parts.

The hydroxyl group-containing acrylic resin (C) that is used according to need is not particularly restricted but resins which are used generally in coating composition field can be used.

The hydroxyl value of the hydroxyl group-containing acrylic resin (C) is preferably 40 to 200 mgKOH/g, more preferably 60 to 120 mgKOH/g. If less than 40 mgKOH/g, the cross-linking reaction site with the curing agent (B) may be deficient and the coating film properties may become insufficient. If over 200 mgKOH/g, the cross-linking reaction site may be too much so that the obtained coating film becomes hard and fragile, or the humidity resistance and water resistance of the coating film may be decreased as a result of excess hydroxyl groups unfavorably.

The weight average molecular weight of the hydroxyl group-containing acrylic resin (C) is preferably 5000 to 70000, more preferably 10000 to 50000. If less than 5000, the properties of the coating film tend to be deteriorated. If over 70000, the coating workability tends to be worse and the finish appearance tends to be deteriorated.

The hydroxyl group-containing acrylic resin (C) can be obtained by polymerizing a monomer composition comprising a hydroxyl group-containing radical polymerizable monomer and a other radical polymerizable monomer to be used according to need by an ordinary method.

The hydroxyl group-containing radical polymerizable monomer is not particularly restricted but includes, for example, 2-hydroxyethyl (meth)acrylate, 4-hydroxybuthyl (meth)acrylate, hydroxypropyl (meth)acrylate, a compound obtained by ring-opening reaction of 2-hydroxyethyl (meth)acrylate by ε-caprolactone (PLACCEL FA series and FM series manufactured by DAICEL CHEMICAL INDUSTRIES, LTD.) and so on. These compounds may be used as single or in combination.

The other radical polymerizable monomer is not particularly restricted but includes, for example, carboxylic acid group-containing monomers such as (meth)acrylic acid, maleic acid, and itaconic acid, epoxy group-containing monomers such as glycidyl (meth)acrylate, methyl(meth)acrylate, ethyl(meth)acrylate, isopropyl (meth)acrylate, n-butyl (meth)acrylate, 2-ethylhexyl(meth)acrylate, styrene, vinyl toluene, vinyl acetate, and α-methylstyrene. These compounds may be used as single or in combination.

The hydroxyl group-containing acrylic resin (C) can be obtained by polymerizing the above-mentioned monomer composition, but a method for producing an acrylic resin known in the prior art may be used for producing the hydroxyl group-containing acrylic resin (C). That is, polymerization methods such as solution polymerization, nonaqueous dispersion polymerization, and bulk polymerization may be used, the solution polymerization method is suitable in terms of easiness of polymerization, control of molecular weight, and conversion to a coating composition.

In the coating composition, it is preferred that the composition contains the polyester resin (A) and the hydroxyl group-containing acrylic resin (C) in the ratio by weight of 100:0 to 40:60. The coating film properties can be obtained sufficiently without losing bio-based content by maintaining within the above-mentioned range.

The coating composition's form is not particularly restricted but includes arbitrary forms such as a solvent coating composition, a water-borne coating composition, and a powder coating composition. The production method thereof is not particularly restricted but the coating compositions can be obtained by known normal production methods.

The coating composition may contain common additive agents used in the coating composition field. The common additive agents includes known additive agents such as coloring pigments, moisture-resistant pigments, other resins, dispersants, anti-setting agents, organic solvents, anti-forming agents, thickening agents, corrosion resistance agents, ultraviolet absorbers, anti-oxidant agents, hindered amine, and surface conditioners.

The coating composition of the present disclosure can be used suitably as a clear coating composition, particularly. Hereinafter, we discuss about the use as a clear coating composition.

The clear coating composition of the present disclosure may be applied to every substrate such as wood, iron, copper, aluminum, tin, zinc, and alloys containing these metals, glass, fabric, plastics, foamed bodies, and molded bodies, for example. In particularly, it can be applied to the surface of plastics and metals. The clear coating composition can be used suitably for an automotive body, and automotive parts including bumper. The clear coating composition can be applied at a time to several articles to be coated comprising different materials respectively, for example the bumper and the automotive body.

When the substrate is a steel plate, the substrate is preferably the one on which an undercoating film, an intermediate coating film, and a base coating film are formed, before the clear coating composition is applied. When the substrate is a metal, the substrate is preferably the one which is chemical treated by phosphates or chromates in advance.

As a method for forming the undercoating film, for example, a method using an electrodeposition coating composition may be mentioned. The electrodeposition coating composition may be a cationic or an anionic type one, but a cationic electrodeposition coating composition is preferred in terms of the corrosion resistance.

The intermediate coating film is formed to obtain the surface smoothness after applying the undercoating composition with covering the base defect (appearance improvement) and to provide good coating film properties (impact resistance, chipping resistance and so on). The intermediate coating film is obtained by using an intermediate coating composition, the intermediate coating composition comprises an organic or a inorganic several coloring pigments, extender pigments, coating film-forming resins, and curing agents, commonly.

As the coating film-forming resin and curing agent, coating film-forming resins such as acrylic resins, polyester resins, alkyd resins, and fluorine resins, and curing agents such as amino resins and/or block polyisocyanate compounds may be used. In view of pigment dispersibility and workability, a combination of alkyd resins and/or polyester resins with amino resins is preferred.

As the intermediate coating composition, a gray intermediate coating composition normally containing a carbon black and titanium dioxide as main pigment is used. In addition, a color intermediate coating composition containing a combination of several coloring pigments such as set gray may be used.

The intermediate coating film may be used with uncured state after the intermediate coating composition is applied to a substrate on which the undercoating film is formed. The curing temperature preferably has 100° C. of the lower limit and 180° C. of the upper limit, when the intermediate coating film is cured. When less than 100° C., curing may be insufficient. When over 180° C., the obtained coating film may be hard and fragile. The lower limit is more preferably 120° C., and the upper limit is more preferably 160° C. By maintaining the curing temperature within the range, a cured coating film having high crosslink density can be obtained. The curing time may be changed according to the curing temperature, and is suitable 10 to 30 minutes at 120 to 160° C.

The base coating film is generally obtained by using a base coating composition comprising a coloring pigment, a coating film-forming resin, and a curing agent, and an additive agent if need arises.

The coloring pigment to be contained in the base coating composition includes coloring agents known in the prior art such as, for example, organic pigments including azo lake pigments, insoluble azo pigments, condensed azo pigments, phthalocyanine pigments, indigo pigments, perinone pigments, perylene pigments, dioxazine pigments, quinacridone pigments, isoindolinone pigments, and metal complex pigments and inorganic pigments including chrome yellow, yellow iron oxide, colcothar, carbon black, and titanium dioxide. In addition, flat pigments including aluminum powder and graphite powder may be added. Extender pigments including calcium carbonate, barium sulfate, clay and talc may be contained. According to need, luster materials such as interference mica pigment and aluminum pigments may be contained.

As the coating film-forming resin and the curing agent in the base coating composition, coating film-forming resins such as acrylic resins, polyester resins, alkyd resins, and fluorine resins and curing agents such as amino resins and/or block polyisocyanate compounds are used.

The total pigment concentration (PWC) in the base coating composition has preferably 3 weight % of the lower limit and 70 weight % of the upper limit. If over 70 weight %, the film appearance deteriorates. The lower limit is more preferably 4 weight %, still more preferably 5 weight %. The upper limit is more preferably 65 weight %, still more preferably 60 weight %.

As the base coating composition, a solution type composition is preferably used in general. If the composition is solution type, it may be organic solvent type, water-borne type (water soluble, water dispersible, and emulsion), or nonaqueous dispersive type.

When the base coating composition is applied to the substrate on which the undercoating film and the intermediate coating film are formed, the base coating film can be obtained by following the same coating procedure as that of the clear coating composition.

The film thickness of the coating film on coating of the base coating composition may be changed according to the desired purpose, but 10 to 30 μm is useful in many cases. When less than 10 μm, the base surface may not be covered to generate a film break. When over 30 μm, the distinctness of image may be deteriorated and problems such as the uniformity and the flow during coating may be occurred. The dried film thickness of the base coating film is generally 10 to 30 μm. When less than 10 μm, the covering property may be deteriorated. When over 30 μm, it is not economical.

A coating film formed by using the base coating composition may be coated by the next clear coating composition without heat curing, but it may be dried at 60 to 120° C. In the case of a water-borne coating composition, the film appearance may be deteriorated due to the blending with the clear coating composition if the drying temperature is 60° C. or less. If the drying temperature is so high, the peeling between the base coating film and the clear coating film may be occurred. The drying time is changed according to the drying temperature, the more preferred drying condition is that the temperature is 80 to 100° C. and the time is 1 to 5 minutes.

The coating method of the substrate using the clear coating composition of the present disclosure is not particularly restricted but includes, for example, spray coating method, electrostatic coating method, and so on. Industrially, there may be mentioned methods using an air electrostatic spray coating machine commonly known as “react gun”, or rotary atomization electrostatic coating machines commonly known as “micro micro bell”, “micro bell”, and “metallic bell”.

The dried film thickness of the clear coating film formed by the clear coating composition is preferably within 10 μm of the lower limit to 70 μm of the upper limit. When less than 10 μm, the base surface may be not covered. When over 70 gm, troubles such as bubbling, sagging and so on may be occurred. The lower limit is more preferably 20 μm, and the upper limit is more preferably 50 μm.

The curing temperature to cure the clear coating film after coating is preferably within 60° C. of the lower limit to 180° C. of the upper limit. When less than 60° C., curing may be insufficient. When over 180° C., the obtained coating film may be fragile. The lower limit is more preferably 80° C. The curing time is changed according to the curing temperature, but 20 to 30 minutes is appropriate at 80 to 160° C.

When the clear coating film is applied onto plastic substrates, it may be applied onto substrates that are coated by common methods such as primer coating, base coating and so on according to need.

### (Adhesive Composition)

The composition containing the polyester resin (A) and a curing agent (B) can be used as an adhesive composition. When used as the adhesive composition, the same curing agent as described in the above-mentioned coating composition can be used in the same proportion as that of the above-mentioned coating composition.

About the adhesive composition of the present disclosure, the purpose thereof is not particularly restricted but includes, for example, an adhesive which is used when a multilayer composite film is produced, and an adhesive which is used for adhering a metallic foil and a metallic plate such as a steel plate, or metal evaporated film to a plastic film such as polypropylene, polyvinyl chloride, polyester, fluorine resin, and acrylic resin.

### (Polyurethane Foam)

The present disclosure relates to a polyurethane foam which is obtained by foaming a composition containing the polyester resin (A) and a polyisocyanate (B-1). That is, the above-mentioned polyester resin can be used as the polyol to be used in the production of the polyurethane foams.

A method for producing the polyurethane foam of the present disclosure is not particularly restricted but the polyurethane foam can be produced by publicly known any method as long as the polyester resin (A) is used. More specifically, for example, the polyurethane foam can be obtained by injecting a composition containing the polyester resin (A), the polyisocyanate (B-1), a catalyst, a foam stabilizers, a foaming agent, and a crosslinking agent to a mold tool, foaming and removing from the mold tool. The polyisocyanate (B-1) can include the compounds that are described as the polyisocyanates to be used in the above-mentioned coating composition.

As the foaming stabilizer, normal surfactants can be used and organosilicon surfactants can be used suitably. For example, B-4113LF manufactured by Goldschmidt Co., Ltd. and L-5309 manufactured by Nippon Unicar Company Limited are mentioned. These may be used as single or in combination. The added amount of the foam stabilizer is preferably 0.01 to 10 weight % relative to the polyester resin (A) to obtain uniform cells.

As the foaming agent, water is used mainly. Water produces carbon dioxide gas by reacting with an isocyanate group and thereby foams the composition. The other foaming agent may be use in addition to water. For example, a small amount of low-boiling organic compounds including cyclopentane and isopentane may be used, and air, nitrogen gas, or liquefied carbon dioxide is mixed in the stock solution by using a gas loading apparatus and dissolved to foam. The added amount of the foaming agent changes depending on the desired density of the obtained article. The amount is usually 0.5 to 15 weight % relative to the polyester resin (A), but is preferably 0.8 to 1.5 weight % for purposes of cushion materials and buffer materials. When the amount is over the upper limit, it may become difficult to stabilize foaming, and when less than the lower limit, the foaming may not be occurred effectively.

The crosslinking agent preferably includes low molecular active hydrogen compounds having the molecular weight less than 500 such as low molecular alcohols, low molecular amines, and low molecular amino alcohols.

These compounds may be used as single or in combination. Among them, low molecular amino alcohols are preferred because the reaction with an isocyanate group is gradual and diethanolamine is especially preferred.

In the production of the polyurethane foam of the present disclosure, publicly known various additives and auxiliary agent such as antistaling agents including antioxidants and ultraviolet absorbers, fillers including calcium carbonate and barium sulfate, fire-retardants, plasticizers, coloring agents, and antifungus agents may be used when needed.

### (Resin Particle)

The polyester resin can be used as a material for resin particles. The resin particles are obtained by crosslinking reaction of resins and curing agents by suspension polymerization. Such resin particles can be used as additives for coating composition, and materials for cosmetics. Further, these resin particles have good elasticity, so a coating film which has good texture can be obtained when it is added in a coating composition and a cosmetic which has good feeling in use can be obtained when it is added in a cosmetic.

Such resin particles can be obtained by reaction of a mixture comprising the above-mentioned polyester resin of the present disclosure, a curing agent (B), and a hydroxyl group-containing acrylic resin (C) that is used according to need under suspension condition. As the hydroxyl group-containing acrylic resin (C) and the curing agent (B) to be used, the same compounds as mentioned in the coating composition can be used. The reaction under suspension condition is conducted by general methods. More specifically, the resin particles can be obtained by reacting at 40 to 80° C. and for 4 to 10 hours. The resin particle preferably has the bio-based content of 25 to 55%.

The number average particle diameter of the resin particles is preferably 2 to 20 μm. The resin particles of which the number average particle diameter is within the above-mentioned range is preferred because they are effective as delustering agents, feeling conditioners in cosmetics, and so on.

The resin particles obtained by the above-mentioned methods may be used as cosmetic materials, and resin particles in matte coating compositions. As the cosmetic materials, the resin particles can be used for any cosmetics including makeup cosmetics such as foundation, rouge, and eye shadow; basic skin cares such as creme, lotion, emulsion, and beauty gel; hair cosmetics such as shampoo, rinse, and hair dressing.

The matte coating composition comprising the resin particles is a coating composition comprising a resin for coating composition, a curing agent to be used if needed, and other additive agents to be added if needed in addition to the resin particles. The resin particle can give matte image to the appearance and obtain the matted appearance. Further, the above-mentioned polyester resin of the present disclosure may be used as at least a part of the resin for the coating composition which forms a matrix in such a matte coating composition.

The resin particles are preferably contained in the proportion of 10 to 40 weight % relative to the total solid matter of the matte coating composition.

### (Acrylic Monomer)

An energy curable unsaturated monomer containing the bio-based structure at high concentration can be obtained by reacting the terminal functional group in the polyester resin of the present disclosure with a polymerizable unsaturated monomer. These acrylic monomers contain the molecular structure expressing the elasticity, so a resin after curing expresses the elasticity to obtain a cured article with good impact resistance.

Acrylic monomers containing 2 or more unsaturated groups can be obtained by esterification reaction of a polyesterpolyol of which the terminal group is hydroxyl group being the polyester resin of the present disclosure with an unsaturated group-containing carboxylic acid compound such as (meth)acrylic acid. There is mentioned a method comprising the reaction of hydroxyl group with an acid anhydride and then addition of a cyclic ether-containing monomer such as glycidyl methacrylate, and a method of adding a hydroxyl group-containing monomer via diisocyanate. In addition, an acrylic monomer containing 2 or more acrylic groups can be obtained by esterification reaction of the polyester resin of the present disclosure of which the terminal group is carboxylic group with hydroxyethyl (meth)acrylate, or by adding a cyclic ether-containing monomer such as glycidyl methacrylate.

The method of reaction is not particularly restricted, but the reaction may be conducted under common reaction condition which are used for these compounds.

The acrylic monomer preferably has the number average molecular weight of 650 to 5200, preferably the bio-based content of 25 to 65 weight %.

### (Energy Curable Coating Composition)

The acrylic monomer may be used as a resin constituting an energy curable coating composition. The energy curable coating composition may contain the above-mentioned acrylic monomer in conjunction with other acrylic monomer. Such the energy curable coating composition of the present disclosure has a structure which expresses the elasticity, so a cured resin has the elasticity and the cured coating film with good impact resistance can be obtained.

The other acrylic monomer includes, for example, compounds containing acrylate functional groups. For example, trimethylolpropane tri(meth)acrylate, pentaerythritol tri(meth)acrylate, pentaerythritol tetra(meth)acrylate, dipentaerythritol hexa(meth)acrylate, dipentaerythritol tetra(meth)acrylate, isocyanuric acid-denatured tri(meth)acrylate, and so on. These (meth)acrylates may be denatured at a part of the molecule structure, and may be denatured by using ethylene oxide, propylene oxide, caprolactone, isocyanuric acid, alkyls, cyclic alkyls, aromatic series, and bisphenols.

Among them, the (meth)acrylate preferably contains 3 or more functional groups in being able to give sufficient hardness to an optical laminate.

In the specification, “(meth)acrylate” means methacrylate and acrylate.

As marketed products of the (meth)acrylate resin that can be used in the present disclosure, there may be mentioned, for example, KAYARAD series and KAYAMER series manufactured by NIPPON KAYAKU Co., Ltd. including DPHA, PET30, TMPTA, DPCA20, DPCA30, DPCA60, and DPCA120; ARONIX series manufactured by TOAGOSEI Co., Ltd. including M315, M305, M309, M310, M313, M320, M325, M350, M360, M402, M408, M450, M7100, M7300K, M8030, M8060, M8100, M8530, M8560, and M9050; NK ester series manufactured by Shin-Nakamura Chemical Co., Ltd. including ADP51, and ADP33; New Frontier series manufactured by DAI-ICHI KOGYO SEIYAKU CO., LTD. including TMPT, TMP3, TMP15, TMP2P, TMP3P, and PETS; Ebecryl series manufactured by Daicel-UCB Company. Ltd. including TMPTA, TMPTAN, PETAK, and DPHA; TMP manufactured by KYOEISYA CHEMICAL Co., Ltd.

The energy curable resin may be, for example, urethane (meth)acrylate containing an acrylate functional group. This can be obtained by the reaction of a polyalcohol, an organic polyisocyanate, and a hydroxyl(meth)acrylate.

As marketed products of the urethane(meth)acrylates that can be used in the present disclosure, there may be mentioned, for example, Shikoh series manufactured by Nippon Synthetic Chemical Industry Co., Ltd. including UV1700B, UV6300B, UV765B, UV7640B, and UV7600B; Art-Resin series manufactured by Negami Chemical Industrial Co., Ltd. including Art-resin HDP, Art-resin HDP-4, Art-resin UN9000H, Art-resin UN3320HA, Art-resin UN3320HB, Art-resin UN3320HC, Art-resin UN3320HS, Art-resin UN901M, Art-resin UN902MS, Art-resin UN903, and Art-resin UN904; UA100H, U4H, U4HA, U6H, U6HA, U15HA, UA32P, U6LPA, U324A, and U9HAMI manufactured by Shin-Nakamura Chemical Co., Ltd.; Ebecryl series manufactured by Daicel-UCB Company. Ltd. including 1290, 5129, 254, 264, 265, 1259, 1264, 4866, 9260, 8210, 204, 205, 6602, 220, and 4450; BEAMSET series manufactured by Arakawa Chemical Industries, Ltd. including BEAMSET 371, and BEAMSET 577; RQ series manufactured by Mitsubishi Rayon Co., Ltd.; UNIDIC series manufactured by DIC Corporation; DPHA40H (manufactured by NIPPON KAYAKU Co., Ltd.), CN9006 (manufactured by Sartomer Technology Company, Inc.), CN968, and so on. Among them, UV1700B, and UV6300B (manufactured by Nippon Synthetic Chemical Industry Co., Ltd.), DPHA40H (manufactured by NIPPON KAYAKU Co., Ltd.), Art-resin HDP and Art-resin UN3320HS (manufactured by Negami Chemical Industrial Co., Ltd.), BEAMSET 371 (manufactured by Arakawa Chemical Industries, Ltd.), BEAMSET 577 (manufactured by Arakawa Chemical Industries, Ltd.), and U15HA and U15H (manufactured by Shin-Nakamura Chemical Co., Ltd.).

The energy curable coating composition preferably satisfies the following relationship that (bio-based acrylic monomer)>(petroleum-based acrylic monomer) on weight basis in the added amount, when bio-based acrylic monomers and petroleum-based acrylic monomers comprising a mineral resource as a material are used in mixture. In the present disclosure, a composition satisfying the relationship can be obtained by using a compound obtained by using a bio-based polyester as an acrylic monomer.

The energy curable coating composition may contain other components in addition the above-mentioned components. The other components may include, for example, photopolymerization initiators, leveling agents, crosslinking agents, curing agents, polymerization accelerators, and viscosity modifiers. These components are not particularly restricted, but known components can be used.

The energy curable coating composition can be applied and cured by the same operation as for normal energy curable coating compositions. It is preferred because it can obtain the same performance as conventional energy curable coating compositions in the properties such as the hardness, and the transparency.

### (A Curable Resin Composition Having A Terminal Isocyanate Group)

The present disclosure relates to a curable resin composition having a terminal isocyanate group obtained by reacting the polyester resin (A) and a polyisocyanate (B-1). More specifically, it is a curable resin composition having a terminal isocyanate group obtained by reacting these two components with an excess of isocyanate groups, removing water under reduced pressure. The polyisocyanate (B-1) to be used in this case may include the compounds which are described in the coating composition.

In the curable resin composition having a terminal isocyanate group, the equivalent ratio (NCO/OH) of isocyanate groups in the polyisocyanate and hydroxyl groups in the polyester polyol is preferably 1.5 to 3.0. As long as the ratio is within this range, the viscosity does not increase remarkably under heating and melting condition for a prolonged time in a fusion apparatus, and there is less foaming caused by carbon dioxide in curing reaction. Further, there is less affect by the volatilization of the unreacted polyfunctional isocyanate compound on the working environment.

The reaction conditions in the above-mentioned reaction are not particularly restricted but the reaction can be conducted following normal methods. The number average molecular weight of the curable resin composition having a terminal isocyanate group is not particularly restricted but is preferably 600 to 6000.

### (Moisture-Curable Reactive Hot-Melt Adhesive Composition)

The moisture-curable reactive hot-melt adhesive composition of the present disclosure is a hot-melt adhesive composition containing the curable resin composition having a terminal isocyanate group.

The initial coagulation power of the moisture-curable reactive hot-melt adhesive composition can be improved by adding a thermoplastic resin having the molecular weight of 30000 or more. The thermoplastic resin includes acrylic resins and so on. The added amount of the thermoplastic resin is preferably 5 to 15 weight % relative to the total amount of the moisture-curable reactive hot-melt adhesive composition. The thermoplastic resin may be added with the polyol in the synthesis of the curable resin composition having a terminal isocyanate group, or after the synthesis of the curable resin composition having a terminal isocyanate group.

The moisture-curable reactive hot-melt adhesive composition of the present disclosure may be contain other components such as a tackifier resin, a catalyst, a nucleation agent, a coloring agent, an antistaling agent, and a thermoplastic resin, if needed. The tackifier resin may include styrene resins, terpene resins, aliphatic petroleum resins, aromatic petroleum resins, and rosin esters. The catalyst may include tertiary amine catalysts and tin catalysts. The nucleation agent may include paraffin wax and microcrystalline wax. It is effective to add a catalyst or a nucleation agent for improved curability under low temperature.

### (Resin Composition Having a Terminal Hydroxyl Group)

The present disclosure relates to a resin composition having a terminal hydroxyl group obtained by reacting the polyester resin (A) and a polyisocyanate (B-1). This resin composition can be used mainly as a print ink. The method for producing the resin is not particularly restricted but may include a method comprising mixing the polyester resin (A) and the polyisocyanate (B-1) in an organic solvent in the mixing proportion that the hydroxyl groups are excess, and then reacting.

### (Print Ink Composition)

The present disclosure relates to a print ink composition containing the resin composition having a terminal hydroxyl group. The print ink composition may be obtained by adding various pigments and solvents to the resin composition having a terminal hydroxyl group, and adding additives such as antiblocking agents and plasticizing agents, pigment dispersants for improving the flow property and the dispersibility and resins such as cellulose resins, maleic acid resins, and polyvinyl butyral if needed, by using well-known pigment dispersers such as sand mill.

As the solvent to be used in the print ink composition of the present disclosure, alcohol solvents and ester solvents being well known as solvents for a print ink can be used. Ketone solvents and aromatic solvents can be used within a scope which does not affect gravure printing and post-process, but the use thereof is restricted because of adverse affects on resin plates to be used in flexo printing.

The alcohol solvent includes, for example, aliphatic alcohols containing 1 to 7 carbon atoms such as methanol, ethanol, normal propanol, isopropanol, n-butanol, isobutanol, and tertiary butanol; glycol ethers such as propylene glycol monomethyl ether, propylene glycol monoethyl ether, propylene glycol propyl ether, propylene glycol isopropyl ether, and propylene glycol monobutyl ether. Among them, alcohol solvents having 1 to 7 carbon atoms are preferred and isopropanol, ethanol, normal propanol, and propylene glycol monomethyl ether are especially preferred. These compounds may be used as single or in combination.

The ester solvents may include methyl acetate, ethyl acetate, n-propyl acetate, isopropyl acetate, n-butyl acetate, and isobutyl acetate.

### (Energy Curable Resin)

The present disclosure relates to an energy curable resin by reacting the polyester resin (A), a compound having an unsaturated group and a functional group which is reactive with an isocyanate group, and a polyisocyanate (B-1). That is, in the energy curable resin, the polyester resin (A) is used as a constituent unit of the energy curable resin containing an unsaturated group. The energy curable resin obtained in such a way may be used for an energy curable adhesive composition.

The compound having an unsaturated group and a functional group which is reactive with an isocyanate group includes, for example, a (meth)acrylate containing a hydroxyl group, an acid halide group, or an epoxy group. The (meth)acrylate containing a hydroxyl group includes, for example, 2-hydroxyethyl (meth)acrylate, 2-hydroxypropyl (meth)acrylate, pentaerythritol tri(meth)acrylate, glycerin di(meth)acrylate, glycidyl group-containing compounds including alkyl glycidyl ether and glycidyl (meth)acrylate, and (meth)acrylate adducts.

The (meth)acrylate containing an acid halide group includes (meth)acrylate chloride and (meth)acrylate bromide.

The (meth)acrylate containing an epoxy group includes (meth)acrylate glycidyl esters. The polyisocyanate (B-1) may include the compounds that are mentioned in the coating composition.

The weight average molecular weight of the energy curable resin obtained by a reaction of the above-mentioned components may be 5000 to 50000, and is preferably 10000 to 30000. When the weight average molecular weight is less than 5000, the adhesive property, the heat resistance, and the humidity resistance may be insufficient. When over 50000, it is not preferred because the viscosity of the energy curable resin may become too high and therefore may reduce the coating workability and the processability.

The energy curable resin having such the molecular weight can provide more effectively an energy curable adhesive composition having especially high adhesive property to a PET film, heat resistance, and high temperature and humidity resistance by combining with a (meth)acrylate monomer and a photo initiator described below.

### (Energy Curable Adhesive Composition)

The present disclosure relates to an energy curable adhesive composition containing the energy curable resin. Such the adhesive can be used for the purpose such as automotive parts and electronic parts.

The energy curable adhesive composition may contain a (meth)acrylate monomer. The (meth)acrylate monomer is used as a solvent of the photo initiator described below. For example, there may be mentioned acryloyl morpholine, dimethylacrylamide, diethylacrylamide, diisopropyl acrylamide, isobornyl (meth)acrylate, dicyclopentenyl acrylate, dicyclopentanyl (meth) acrylate, dicyclopentenyl oxyethyl (meth)acrylate, methyl (meth)acrylate, ethyl (meth)acrylate, cyclohexyl methacrylate, dicyclopentadienyl (meth)acrylate, tricyclodecanyl (meth)acrylate, diacetone acrylamide, isobutoxymethyl (meth) acrylamide, 3-hydroxycyclohexyl acrylate, 2-acryloyl cyclohexylsuccinate, and N-vinyl pyrrolidone. Among them, acryloyl morpholine, dimethylacrylamide, and N-vinyl pyrrolidone are especially preferred because these compounds may provide an energy curable adhesive composition being superior in adhesive property. The (meth)acrylate monomer may be used as single or in combination.

The (meth)acrylate monomer is available as a marketed product. As the marketed product, AMO (acryloyl morpholine), LIGHT-ACRYLATE IB-XA (isobornyl acrylate) LIGHT-ACRYLATE IMA (isomyristyl acrylate), manufactured by KYOEISYA CHEMICAL Co., Ltd. are mentioned.

The energy curable adhesive composition may contain a photo initiator.

In the energy curable adhesive composition, the proportion of each component may be set that the energy curable resin: the (meth)acrylate monomer=90:10 to 10:90 (weight parts). The amount of photo initiator may be 0.1 to 10 weight parts relative to 100 weight parts of the total these components.

When the amount of the photo initiator is less than 0.1 weight part relative to the total amount 100 weight parts of the energy curable resin and the (meth)acrylate monomer, it is not preferred because sufficient polymerization initiation property may not be exerted in time of energy curing. When the amount of the photo initiator is over 10 weight parts, it is not preferred because the termination reaction is increased, which leads to lower crosslinking density.

It is possible to add a silane coupling agent as an adhesion accelerator or an epoxy group-containing compound for improving the adhesive property to the energy curable adhesive composition of the present disclosure.

The energy curable adhesive composition may contain a little amount of other component than the additives such as ultraviolet absorbers, antistaling agents, and dyes. Sometimes the energy curable adhesive composition may contain a little amount of fillers such as silica gel, calcium carbonate, and silicone copolymers.

It is needed to choose a method which can form an uniformly-thick layer as the method for applying the energy curable adhesive composition, and known methods including screen coating, spray coating, and dipping coating can be used.

Hereinafter, the present disclosure will be described in more detail by way of examples, but the present disclosure is not limited to these examples. In examples, “part” and “%” mean “weight part” and “weight %” respectively, unless otherwise specified.

### Synthesis Example 1

Trimethylolpropane 135 g, neopentyl glycol 156 g, sebacic acid 404 g, xylene 42 g, and p-toluenesulfonic acid 1.2 g were put into a 1 L-separable flask equipped with a temperature controller, a stirring blade, a nitrogen gas inlet, Dean-Stark trap, and a reflux condenser. The Dean-Stark trap was filled up with xylene to the upper limit. Under nitrogen gas flow, the system inside was heated to 140° C. and maintained for an hour, and then heated to 195° C. to maintain the condensation reaction for 5 hours. After confirming that resin acid value reached 4 mgKOH/g (resin solid matter), the cooling was started. After cooling, the solid fraction was adjusted to 75% by adding butyl acetate.

### Synthesis Examples 2 to 8

Condensation reactions were conducted in compositions shown in the table 1 by following the same procedure as that of synthesis example 1.

TABLE 1

| | | TMP | PE | BD | NPG | MCT | SEA | SA | ND | MPD |
|---|---|---|---|---|---|---|---|---|---|---|
| Synthesis | 1 | 135 | | | 156 | | 404 | | | |
| example | 2 | 109 | | 146 | | | 369 | | | |
| | 3 | 116 | | 134 | | | 324 | 57 | | |
| | 4 | | 82 | 142 | | | 454 | | | |
| | 5 | | | 156 | | 120 | 322 | | | |
| | 6 | | | | | | 337 | | | 263 |
| | 7 | | | | | | | 250 | 243 | 107 |
| | 8 | | | 215 | | | 385 | | | |

TMP Trimethylol propane
PE Pentaerythritol
BD 1,4-butanediol
NPG Neopentyl glycol
MCT Methylcyclohexene tricarboxylic acid
SEA Sebacic acid
SA Succinic acid
ND 1,9-nonane diol
MPD 3-methyl-1,5-pentanediol

### Synthesis Example 9

The same reaction as that of synthesis example 1 in 2 L-flask before the dilution using butyl acetate was conducted, and the mixture was cooled to 90° C. Succinic anhydride 39.3 g was added into it to maintain the reaction for 4 hours until the acid value reached to the desired level, and the solid fraction was adjusted to 75% by adding butyl acetate. Triethyl amine 43.7 g was added to homogenize the mixture and then ion exchanged water 759.93 g was added slowly to phase inversion emulsify. Then, the mixture was put in another flask and the solid matter thereof was adjusted to 30%. Emulsion having the particle diameter of 95 nm and being stable was obtained.

The properties of polyester resins obtained in synthesis examples 1 to 9 were shown in table 2.

TABLE 2

| | | Number average | Hydroxyl | Acid value | Bio-based | Crystal melting |
|---|---|---|---|---|---|---|
| | | molecular weight | group value | (mgKOH/g) | content (%) | heat (cal/g) |
| Synthesis | 1 | 1320 | 184.1 | — | 58.1 | 1.0 or less |
| example | 2 | 1260 | 204.1 | — | 82.5 | 1.0 or less |
| | 3 | 2020 | 142.2 | — | 81.6 | 1.0 or less |
| | 4 | 2460 | 101.9 | — | 87.9 | 1.0 or less |
| | 5 | 1970 | — | 143.6 | 79.9 | 1.0 or less |
| | 6 | 1100 | 104 | — | 56.2 | 1.0 or less |
| | 7 | 1980 | 57 | — | 41.7 | 1.0 or less |
| | 8 | 1280 | 89 | — | 100 | 80 |
| | 9 | 1390 | 172 | 30 | 85.1 | 1.0 or less |

### Synthesis Example 10

Butyl acetate 206.8 g was put into a 1 L-separable flask equipped with a temperature controller, a stirring blade, a nitrogen gas inlet, a dropping funnel, and a reflux pipe and heated to 105° C. A mixture containing PLACCEL FM-2 (manufactured by DAICEL CHEMICAL INDUSTRIES, LTD.) 114.1 g, hydroxyethyl methacrylate 111.6 g, methacrylic acid 3.1 g, methyl methacrylate 12.7 g, n-butyl methacrylate 71.8 g, isobornyl methacrylate 126.7 g, and butyl acetate 73.3 g was added by drops for 1 hour. After being maintained at 105° C. for a hour, the mixture was cooled. The number average molecular weight measured by GPC was 3840 and the hydroxyl group value was 150.

### Synthesis Example 11

Xylene 200 g and butyl acetate 150 g were put into the same reaction vessel as that of synthesis example 10 and heated to 90° C. to keep the temperature constant. Next, a mixture of styrene 100 g, 2-ethylhexyl methacrylate 136 g, hydroxyethyl methacrylate 160 g, methacrylic acid 4 g, and azobisisobutyronitrile 16 g was added by drops for 3 hours. After the reaction was maintained for a hour, a mixture of butyl acetate 50 g, and azobisisobutyronitrile 1.6 g was added by drops for 30 minutes and the reaction was maintained for another hour. The number average molecular weight measured by GPC was 6640 and the hydroxyl group value was 172.

### Synthesis Example 12

A mixed aqueous solution of PVA-217EE manufactured by KURARAY CO., LTD., 10 g and ion exchanged water 200 g was produced in the same reaction vessel as that of synthesis example 10. A mixture of the polyester of synthesis example 488.7 g, isophorone diisocyanate 33.5 g, toluene 27.8 g, and dibutyltin dilaurate 0.15 g was added and then mixed at 5000 rpm for 2 minutes by a homogenizer. Ion exchanged water 150 g was added to dilute and the mixture was put into the same reaction vessel as that of synthesis example 9.

After keeping the temperature at 40° C. for 2 hours, the temperature was raised to 75° C. and the reaction was maintained for 6 hours. After cooling, the reactant was dried by spray-drying method to obtain powder polyurethane particles. The value of volume average particle diameter measured by Coulter counter was 8.8 micrometer, and the bio-based content was 54.3%.

### Synthesis Example 13

A mixed aqueous solution of PVA-217EE manufactured by KURARAY CO., LTD., 10 g and ion exchanged water 200 g was produced in the same reaction vessel as that of synthesis example 10. A mixture of the polyester of synthesis example 382.2 g, Duranate TPA-100 manufactured by Asahi Kasei Corporation. 3 8.9 g, toluene 28.9 g, and dibutyltin dilaurate 0.15 g was added and then mixed at 5000 rpm for 2 minutes by a homogenizer. Ion exchanged water 150 g was added to dilute and the mixture was put into the same reaction vessel as that of synthesis example 1. After keeping the temperature at 40° C. for 2 hours, the temperature was raised to 75° C. and the reaction was maintained for 6 hours. After cooling, the reactant was dried by spray-drying method to obtain powder polyurethane particles.

The value of volume average particle diameter measured by Coulter counter was 7.9 micrometer, and the bio-based content was 53.7%.

### Synthesis Example 14

A mixed aqueous solution of PVA-217EE manufactured by KURARAY CO., LTD., 10 g and ion exchanged water 200 g was produced in the same reaction vessel as that of synthesis example 10. A mixture of polyester diol (P-1010 manufactured by KURARAY CO., LTD.,) 59.3 g, Duranate TPA-100 40.7 g, toluene 50 g, and dibutyltin dilaurate 0.15 g was added and then mixed at 5000 rpm for 2 minutes by a homogenizer.

Ion exchanged water 150 g was added to dilute and the mixture was put into the same reaction vessel as that of synthesis example 1. After keeping the temperature at 40° C. for 2 hours, the temperature was raised to 75° C. and the reaction was maintained for 6 hours. After cooling, the reactant was dried by spray-drying method to obtain powder polyurethane particles. The value of volume average particle diameter measured by Coulter counter was 8.6 micrometer, and the bio-based content was 0%.

### Synthesis Example 15

A mixed aqueous solution of PVA-217EE manufactured by KURARAY CO., LTD., 7.5 g, PVA-417 manufactured by KURARAY CO., LTD., 2.5 g and ion exchanged water 200 g was produced in the same reaction vessel as that of synthesis example 10. A mixture of the polyester obtained in synthesis example 6 104.7 g, hexamethylene diisocyanate 21.5 g, toluene 23.8 g, and dibutyltin dilaurate 0.15 g was added and then mixed at 5000 rpm for 2 minutes by a homogenizer. Ion exchanged water 150 g was added to dilute and the mixture was put into the same reaction vessel as that of synthesis example 9. After keeping the temperature at 40° C. for 2 hours, the temperature was raised to 75° C. and the reaction was maintained for 6 hours. After cooling, the reactant was dried by spray-drying method to obtain powder polyurethane particles. The value of volume average particle diameter measured by Coulter counter was 7.6 micrometer, and the bio-based content was 44.1%.

### Synthesis Example 16

The resin solution 400 g obtained in synthesis example 1, succinic anhydride 93.5 g, and butyl acetate 31.2 g were put into the same reaction vessel as that of synthesis example 10. Next, triethylamine 9.35 g was added and the mixture was heated to 90° C. Then, glycidyl methacrylate 126.13 g, and butyl acetate 42.044 g were added by drops for 2 hours and the reaction was maintained for 6 hours. The bio-based content was 64.5%.

### Synthesis Example 17

The same condensation reaction as that of Example 2 before the dilution using butyl acetate was conducted, and the mixture was cooled to 100° C. with the temperature kept constant. Acrylic acid 327 g, p-toluenesulfonic acid 3.3 g, and methoxy hydroquinone 0.95 g were added into it and the reaction was maintained for 8 hours until the acid value reached to the desired level. After xylene and unreacted acrylic acid were removed by reducing the pressure, the solid fraction was adjusted to 75% by adding butyl acetate. Then, the obtained resin solution and added ion-exchanged water 400 g were mixed for 1 hour to remove the aqueous phase. The bio-based content was 65.4%.

### Synthesis Example 18

The same reaction as that of synthesis example 5 was conducted and the temperature was kept at 90° C. Glycidyl methacrylate 203 g, tetrabutylammonium bromide 0.4 g, and butyl acetate 87 g were added into it and the reaction was maintained for 6 hours until the acid value reached to the desired level. The bio-based content was 59.7%.

### Synthesis Example 19

Butyl acetate 300 g was put into a 1 L-separable flask equipped with a temperature controller, a stirring blade, a nitrogen gas inlet, a dropping funnel, and a reflux pipe and heated to 100° C. A mixture of cyclohexyl methacrylate 144.0 g, 2-ethylhexyl methacrylate 72.0 g, methacrylic acid 160.0 g, styrene 24.0 g, azobisisobutyronitrile 10.0 g, and butyl acetate 60.0 g was added by drops for 3 hours. One hour after the dropping, a mixture of azobisisobutyronitrile 1.0 g and butyl acetate 40.0 g was added by drops for 1 hour and the polymerization was continued for 1 hour. Next, the temperature was raised to 115° C. and kept for 3 hours to make the degradation of azobisisobutyronitrile perfect. Methoxy hydroquinone 16 g, and triethylamine 12 g were added and, further glycidyl methacrylate 185 g and butyl acetate 185 g were added by drops for 2 hours. After continuing the reaction for 4 hours, the acid value was measured and the value was 54 mgKOH/g. From this result, it was indicated that almost all of glycidyl methacrylate were reacted. The number average molecular weight was 14800.

### (Evaluation Items and Evaluation Methods of the Coating Film)

### (Initial Adhesion)

It was evaluated according to JIS-K-5600-5-6. Specifically, 2×2 mm 100 squares were made on the coating film by using a cutter knife, an adhesive cellophane tape was attached perfectly, and then an end of the tape was lifted and peeled off upward. These peeling operation was conducted three times at the same point, the number of squares of which 50% or more area were peeled off was shown. 0 was passable (◯), and 1 or more was rejection (x)

### (Humidity Resistance)

It was evaluated according to JIS-K-5600-7-12. Specifically, the test object was left at the temperature of 50±2° C., and humidity of 98±2% for 24 hours, and the coating film was observed at the surface thereof and the square adhesion test was conducted. The square adhesion test comprised forming 100 squares of 2 mm on the coating film by using a cutter knife, attaching an adhesive cellophane tape perfectly thereon, and lifting the end of the tape upward followed by peeling off. These peeling operations were conducted at the same point, and the number of squares in which 50% or more area of the coating film was peeled off was shown.

◯: There was no coating film surface trouble such as whitening and swelling, and the number of peeled off points was 0.
x: There was some coating film surface trouble such as whitening and swelling, or the number of peeled off points was 1 or more.

### (Alkali Resistance)

It was evaluated according to JIS-K-5600-6-1. Specifically, a cylindrical ring was arranged on the coating film surface, 0.1N sodium hydroxide solution 5 mL was added and put a glass plate over the ring. It was left at 55° C. for 4 hours. After water washing, the coating film surface was observed.

◯: There was no coating film surface trouble such as whitening and swelling.
x: There was some coating film surface trouble such as whitening and swelling.

### (Water Resistance)

It was evaluated according to JIS-K-5600-6-1.

Specifically, a cylindrical ring was arranged on the coating film surface, distillated water 5 mL was added and put a glass plate over the ring. It was left at 55° C. for 4 hours. After water washing, the coating film surface was observed.

◯: There was no coating film surface trouble such as whitening and swelling.
x: There was some coating film surface trouble such as whitening and swelling.

### (Acid Resistance)

It was evaluated according to JIS-K-5600-6-1. Specifically, a cylindrical ring was arranged on the coating film surface, 0.1N sulfuric acid solution 5 mL was added and put a glass plate over the ring. It was left at 55° C. for 4 hours. After water washing, the coating film surface was observed.

◯: There was no coating film surface trouble such as whitening and swelling.
x: There was some coating film surface trouble such as whitening and swelling.

### (Scratching Resistance)

The scratching level of the coating film surface was observed by eye after the coating film surface was rubbed with a steel wool #1000 back and forth 20 times.

◯: There was almost no scratching.
Δ: There was a few scratching.
x: There was many scratching.

### (Appearance)

It was observed by eyes.

◯: There was no surface defect of the coating film such as swelling, breaking, and a pin hole.
x: There were some surface defects of the coating film such as swelling, breaking, and a pin hole.

### (Bio-Based Content)

It was calculated from the proportion of the bio-based materials in the bio-based curable materials.

### Example 1

The resin solution 66.0 g of synthesis example 1, Duranate TPA-100 manufactured Asahi Kasei Corporation. 34.0 g, butyl acetate 67.0 g, BYK-310 (manufactured by BYK) 1.67 g, and dibutyltin dilaurate 0.013 g were mixed until translucent uniformly and spray coated on ABS substrate in such a way that the film thickness was 30±3 μm. After coating and leaving at room temperature for 10 minutes, the temperature of the coating film was raised to 100° C. and the temperature was kept at this point for 30 minutes to obtain a test plate of example 1. The evaluation of the coating film was conducted 24 hours after drying.

### Examples 2 to 9 and Comparative Examples 1 to 3

The coating compositions were prepared and evaluated by following the same procedure as that of example 1. The compositions including example 1 were shown in Table 3. The evaluation results of the coating films were shown in Table 4. In the table, JER 152 is the epoxy resin manufactured by Japan Epoxy Resins Co. Ltd. and Bayhydur 305 is the water-borne polyurethane manufactured by Sumika Bayer Urethane Co., Ltd.

TABLE 3

| | Example | | | | | | | | | Comparative example | | |
|---|---|---|---|---|---|---|---|---|---|---|---|---|
| | 1 | 2 | 3 | 4 | 5 | 6 | 7 | 8 | 9 | 1 | 2 | 3 |
| Resin solution of | 66.0 | 46.7 | | | | | | | | 9.1 | | |
| synthesis example 1 | | | | | | | | | | | | |
| Resin solution of | | | 63.6 | | | | | | | | | |
| synthesis example 2 | | | | | | | | | | | | |
| Resin solution of | | | | 71.5 | | | | | | | | |
| synthesis example 3 | | | | | | | | | | | | |
| Resin solution of | | | | | 77.8 | | | | | | | |
| synthesis example 4 | | | | | | | | | | | | |
| Resin solution of | | | | | | 72.8 | | | | | | |
| synthesis example 5 | | | | | | | | | | | | |
| Resin solution of | | | | | | | | 49.2 | | | | |
| synthesis example 6 | | | | | | | | | | | | |
| Resin solution of | | | | | | | | | 52.6 | | | |
| synthesis example 7 | | | | | | | | | | | | |
| Resin solution of | | | | | | | | | | | | 50.2 |
| synthesis example 8 | | | | | | | | | | | | |
| Resin solution of | | | | | | | 78.4 | | | | | |
| synthesis example 9 | | | | | | | | | | | | |
| Resin solution of | | 25.0 | | | | | | | | 64.5 | 74.8 | |
| synthesis example 10 | | | | | | | | | | | | |
| Resin solution of | | | | | | | | 26.3 | 28.2 | | | 26.9 |
| synthesis example 11 | | | | | | | | | | | | |
| Duranate TPA-100 | 34.0 | 31.2 | 36.4 | 28.5 | 22.2 | | | 24.5 | 19.3 | 26.4 | 25.2 | 22.9 |
| Bayhydur 305 | | | | | | | 21.6 | | | | | |
| JER152 | | | | | | 27.2 | | | | | | |
| Butyl acetate | 67.0 | 59.5 | 84.1 | 82.1 | 80.6 | 81.8 | | 54.3 | 51.2 | 61.5 | 70.1 | 53.4 |
| Ion exchanged water | | | | | | | 45.1 | | | | | |
| BYK-310 | 1.67 | 1.62 | 1.21 | 1.11 | 1.03 | 1.82 | | 1.54 | 1.51 | 1.52 | 1.70 | 1.53 |
| POLYFLOW KL245 | | | | | | | 1.45 | | | | | |
| Dibutyltin dilaurate | 0.013 | 0.012 | 0.013 | 0.012 | 0.012 | | 0.003 | 0.012 | 0.011 | 0.011 | 0.012 | 0.012 |
| Triphenylphosphine | | | | | | 0.182 | | | | | | |

TABLE 4

| | Example | | | | | | | | | Comparative Example | | |
|---|---|---|---|---|---|---|---|---|---|---|---|---|
| | 1 | 2 | 3 | 4 | 5 | 6 | 7 | 8 | 9 | 1 | 2 | 3 |
| Initial adhesion | ◯ | ◯ | ◯ | ◯ | ◯ | ◯ | ◯ | ◯ | ◯ | ◯ | ◯ | Imponderable |
| Humidity resistance | ◯ | ◯ | ◯ | ◯ | ◯ | ◯ | ◯ | ◯ | ◯ | ◯ | ◯ | because of |
| Alkali resistance | ◯ | ◯ | ◯ | ◯ | ◯ | ◯ | ◯ | ◯ | ◯ | ◯ | ◯ | coating |
| Water resistance | ◯ | ◯ | ◯ | ◯ | ◯ | ◯ | ◯ | ◯ | ◯ | ◯ | ◯ | composition |
| Acid resistance | ◯ | ◯ | ◯ | ◯ | ◯ | ◯ | ◯ | ◯ | ◯ | ◯ | ◯ | turbidity |
| Scratching resistance | ◯ | ◯ | ◯ | ◯ | ◯ | ◯ | ◯ | ◯ | ◯ | Δ | Δ | |
| Appearance | ◯ | ◯ | ◯ | ◯ | ◯ | ◯ | ◯ | ◯ | ◯ | X | X | |
| Bio-based content of | 34.4 | 34.7 | 46.8 | 53.3 | 63.7 | 53.3 | 44.4 | 26.9 | 21.7 | 7.7 | 0.0 | |
| coating film | | | | | | | | | | | | |

From the results in Table 4, it was appeared that the coating compositions of the present disclosure were superior in various properties.

### Example 10

### Preparation of Pigment Paste

Acrylic resin varnish BAR 007 (weight average molecular weight 50000; solid matter hydroxyl group value 140; glass transition temperature −20° C.; solid matter 65%,; manufactured by NIPPON BEE CHEMICAL CO., LTD.) 159 parts, xylene 111 parts, coloring pigment monarch 1300 (black pigment; manufactured by Cabot Specialty Chemicals, Inc.) 30 parts were put into a vessel equipped with a stirring apparatus and stirred for 30 minutes. Then, the obtained solution (called mil base) was dispersed by sand grinder mill to prepare black pigment paste. The black pigment concentration in the paste was 10%.

### (Preparation of Coating Composition)

Acrylic resin varnish BAR 007 36.4 g, the polyester of synthesis example 331.3 g, the resin particles of synthesis example 12 36.0 g, delustering agent (Art Pearl C-800 manufactured by Negami Chemical Industrial Co., Ltd.) 13.6 g, the black pigment paste 4.2 g, a curing agent (Desmodur TPLS 2010 manufactured by Bayer Holding Ltd.) 37.5 g were mixed until uniform. The obtained coating composition was diluted by diluting thinner containing butyl acetate/ethyl-3-ethoxypropionate=50 part/50 part so that a diluted coating solution having the viscosity of 15 sec. at 20° C. by #4 Fird Cup viscometer was obtained. The coating solution was applied by air spray on ABS substrate in such a setting that the film thickness is 35 μm and bake cured in drying oven at 80° C. for 30 minutes to obtain a test plate. The evaluation of the coating film was conducted 24 hours after drying.

### Example 11

Polyester dispersed in water of synthesis example 6 100.0 g, polyurethane dispersion (ADEKA BONTIGHTER HUX 561 manufactured by ADEKA CORPORATION) 80.0 g, polyolefin emulsion (Hardlen NZ-1004E manufactured by Toyo Kasei kogyo) 166.7 g, POLYFLOW KL245 (manufactured by KYOEISHA CHEMICAL Co., LTD.) 5 g, polyethylene wax (MPP620VF manufactured by Micropowders) 5 g, buthyl cellosolve 30 g, resin particles of synthesis example 1540 g, FCW black 420 pigment paste (manufactured by NIPPON PAINT Co., Ltd) 25.3 g, PRIMAL ASE 60 (manufactured by Rohm and Haas Company) 10.7 g, and ion exchanged water 10.5 g were mixed until uniform. The obtained water-borne coating composition was spray coated on a polypropylene material, left at room temperature for 5 minutes, and baked at 80° C. for 20 minutes to obtain a test piece having dried film thickness of 25 μm.

### Comparative Example 4

Acrylic resin varnish BAR 007 72.9 g, resin particles of synthesis example 1012.0 g, resin particles of synthesis example 924.0 g, delustering agent (SILK PROTEIN POWDER GSF manufactured by Idemitsu Technofine Co., Ltd.) 13.56 g, black pigment paste of Example 84.2 g, a curing agent (Desmodur TPLS 2010 manufactured by Sumika Bayer Urethane Co., Ltd.) 37.5 g were mixed until uniform. A test piece was formed following the same procedure as that of example 10.

### Comparative Example 5

Acrylic resin varnish BAR 007 72.9 g, resin particles of synthesis example 836.0 g, delustering agent (SILK PROTEIN POWDER GSF manufactured by Idemitsu Technofine Co., Ltd.) 13.56 g, black pigment paste of Example 84.2 g, a curing agent (Desmodur TPLS 2010 manufactured by Sumika Bayer Urethane Co., Ltd.) 37.5 g were mixed until uniform. A test piece was formed following the same procedure as that of example 10.

The results of evaluations for the coating films were shown in table 5.

TABLE 5

| | Exam- | Exam- | Comparative | Comparative |
|---|---|---|---|---|
| | ple 10 | ple 11 | example 4 | example 5 |
| Initial adhesion | ◯ | ◯ | ◯ | ◯ |
| Humidity resistance | ◯ | ◯ | X | X |
| Alkali resistance | ◯ | ◯ | ◯ | ◯ |
| Bio-based content | 33.7 | 29.7 | 4.8 | 0.0 |
| of coating film | | | | |

From the results of table 5, it was apparent that the matte coating compositions of the present disclosure demonstrated superior performance.

### Example 12

UV-curable oligomer of synthesis example 1680 g, Ditrimethylol propane tetraacrylate (Aronix M-408 manufactured by TOA GOSEI CO., LTD.) 20 g, light-curable resin of synthesis example 1940 g, Irgacure 184 (manufactured by BASF) 5 g, butyl acetate 60 g, TINUVIN 400 (manufactured by BASF) 1 g, and BYK333 (manufactured by BYK) 0.2 g were mixed until uniform and spray coated on ABS and PMMA substrate in such a way that the film thickness was 20±3 μm. After coating, the substrate was left at room temperature for 10 minutes and heat treated in a oven at 80° C. for 3 minutes to volatilize organic solvent matter. Then, an energy of which the integrated light quantity was 400 mj/cm² at a wavelength of 340 to 380 nm was irradiated to obtain a cured coating film. The evaluation for the coating film was conducted 24 hours after drying. In example 13 and 14, and Comparative example 6, coating compositions were prepared and evaluated following the same procedure as that of example 12. The compositions including example 12 were shown in table 6.

TABLE 6

| | Exam- | Exam- | Exam- | Comparative |
|---|---|---|---|---|
| | ple 12 | ple 13 | ple 14 | example 6 |
| Resin solution of | 80.0 | | | |
| synthesis example 16 | | | | |
| Resin solution of | | 100.0 | | |
| synthesis example 17 | | | | |
| Resin solution of | | | 80.0 | |
| synthesis example 18 | | | | |
| Resin solution of | 40.0 | 50.0 | 40.0 | 50.0 |
| synthesis example 19 | | | | |
| Aronix M-408 | 20.0 | | 20.0 | 75.0 |
| Irgacure 184 | 5.0 | 5.0 | 5.0 | 5.0 |
| Butyl acetate | 60.0 | 50.0 | 60.0 | 75.0 |
| Tinuvin 400 | 2.0 | 2.0 | 2.0 | 2.0 |
| Tinuvin 292 | 1.0 | 1.0 | 1.0 | 1.0 |
| BYK333 | 0.2 | 0.2 | 0.2 | 0.2 |

The results of evaluations for the coating films were shown in table 7.

TABLE 7

| | | | | Comparative | | | | Comparative |
|---|---|---|---|---|---|---|---|---|
| | Example 12 | Example 13 | Example 14 | example 6 | Example 12 | Example 13 | Example 14 | example 6 |
| Substrate | ABS | ABS | ABS | ABS | PMMA | PMMA | PMMA | PMMA |
| Initial adhesion | ◯ | ◯ | ◯ | ◯ | ◯ | ◯ | ◯ | X |
| Humidity resistance | ◯ | ◯ | ◯ | ◯ | ◯ | ◯ | ◯ | ◯ |
| Alkali resistance | ◯ | ◯ | ◯ | ◯ | ◯ | ◯ | ◯ | ◯ |
| Water resistance | ◯ | ◯ | ◯ | ◯ | ◯ | ◯ | ◯ | ◯ |
| Acid resistance | ◯ | ◯ | ◯ | ◯ | ◯ | ◯ | ◯ | ◯ |
| Scratching resistance | ◯ | ◯ | ◯ | ◯ | ◯ | ◯ | ◯ | ◯ |
| Appearance | ◯ | ◯ | ◯ | ◯ | ◯ | ◯ | ◯ | X |
| Bio-based content of | 38.7 | 49.1 | 35.8 | 0 | 38.7 | 49.1 | 35.8 | 0 |
| coating film | | | | | | | | |

From the results of table 7, it was apparent that the matte coating compositions of the present disclosure demonstrated superior performance.

### Examples 15, 16 and Comparative Example 7

The oil components 1 to 5 in table 9 were mixed, heated to 90° C. to dissolve the components uniformly, and the temperature was kept at 70° C. Next, the water-borne components 6 to 9 in table 9 were mixed and heated to 80° C. The components 10 to 15 in table 9 were added to the mixture and dispersed uniformly, and then the temperature was kept at 70° C. The oil components prepared in advance was added gradually under stirring to emulsify, and cooled to the room temperature to obtain a sun block lotion. The sun block lotions were evaluated by 30 panelists on a scale of 1 to 5 shown in table 8, and the evaluation results were shown in table 9. The unit in the table is gram (g).

TABLE 8

| Fairly good | Good | Normal | Bad | Significantly worse |
|---|---|---|---|---|
| 5 | 4 | 3 | 2 | 1 |

TABLE 9

| | | Exam- | Exam- | Comparative |
|---|---|---|---|---|
| | | ple 15 | ple 15 | example 7 |
| 1 | Stearic acid | 0.5 | 0.5 | 0.5 |
| 2 | Cetyl alcohol | 1.5 | 1.5 | 1.5 |
| 3 | Vaseline | 3.4 | 3.4 | 3.4 |
| 4 | Liquid paraffin | 6.9 | 6.9 | 6.9 |
| 5 | Homooxyethylene sorbitan | 1.5 | 1.5 | 1.5 |
| | monostearate | | | |
| 6 | Glycerin | 2.5 | 2.5 | 2.5 |
| 7 | Methyl parahydroxybenzoate | 0.1 | 0.1 | 0.1 |
| 8 | Potassium hydrate | 0.1 | 0.1 | 0.1 |
| 9 | Purified water | 63.9 | 63.9 | 63.9 |
| 10 | Titanium oxide | 15 | 15 | 15 |
| 11 | Bio-based resin particles | 10 | | |
| | of synthesis example 13 | | | |
| 12 | Bio-based resin particles | | 10 | |
| | of synthesis example 15 | | | |
| 13 | Petroleum-based resin parti- | | | 10 |
| | cles of synthesis example 14 | | | |
| 14 | Perfume | 0.01 | | |
| 15 | Antiseptic agent | 0.01 | | |
| Results | Soft and smooth usability | 4.5 | 4.6 | 3.5 |
| of | Extension property | 4.2 | 4.6 | 3.3 |
| evalu- | Fresh-feeling after use | 4.7 | 4.3 | 3.6 |
| ation | Transparency | 4.6 | 4.5 | 3.6 |
| | Comprehensive evaluation | 4.5 | 4.5 | 3.5 |

From the results in table 9, the cosmetics of the present disclosure had superior properties.

### Synthesis Examples 20 to 22

The components shown in table 10 were mixed in the same reaction vessel as that of synthesis example 1, and the reaction was conducted at 120° C. for 5 hours. A resin solution was obtained after the disappearance of the isocyanate groups was confirmed by IR spectroscopy. The unit in table 10 is gram (g).

TABLE 10

| | Synthesis | Synthesis | Synthesis |
|---|---|---|---|
| | example 20 | example 21 | example 22 |
| Resin solution of | 200.0 | | |
| synthesis example 1 | | | |
| Resin solution of | | 200.0 | |
| synthesis example 6 | | | |
| Resin solution of | | | 200.0 |
| synthesis example 7 | | | |
| Isophorone | 7.8 | 15.4 | 8.5 |
| diisocyanate | | | |
| Butyl acetate | 318.2 | 336.0 | 319.7 |

### Example 17

### Preparation and Evaluation of Gravure Ink

The resin solution 120 parts obtained in synthesis example 20, beta phthalocyanine pigment 30 parts, polyethylene wax 3 parts, isopropyl alcohol 30 parts, and ethyl acetate 120 parts were mixed and dispersed by using a horizontal sand mill to prepare a gravure print ink. The obtained print ink was adjusted by using a mixed solvent containing ethyl acetate and isopropyl alcohol (weight ratio=40:60) in such a way that the viscosity thereof was 18 seconds when it was measured by using the Zahn cup No. 3. Then, the gravure ink was printed on a corona treated stretched polypropylene film and a corona treated polyester film by a gravure printing machine equipped with a 175 lines/inch helio plate and dried at 50° C. to obtain a printed film. The printed film was evaluated about adhesion property by a tape adhering test and blocking resistance. The results were shown in table 11.

### (Tape Adhering Test)

Cello tape (trademark) was adhered to the printed side and smeared the tape side with thumb five times to compress. Then, the tape was peeled off in direction at right angles to the printed side and the condition of the ink film was observed.

### Decision Criterion

◯: 75% or more ink remained on the film.
Δ: Over 30% but less than 75% ink remained on the film.
x: Ink less than 30% remained on the film.

### (Blocking Resistance Test)

Two films were stuck together at their printed side and kept at 40° C., 80% relative humidity, and 10 kgf/cm² for 24 hours. After then, the transition degree to the attached ink side was judged according to the following decision criterion at room temperature.

◯: The transition amount of ink was less than 10%
Δ: The transition amount of ink was 10 to 30%
x: The transition amount of ink was over 30%

### Comparative Example 8

A gravure ink was prepared and evaluated following the same procedure as that of Example 17 using the resin obtained in synthesis example 21. The evaluation result was shown in table 11.

### Comparative Example 9

A gravure ink was prepared and evaluated following the same procedure as that of Example 17 using the resin obtained in synthesis example 22. The evaluation result was shown in table 11. The bio-based content was the value about the solid matter in the ink.

TABLE 11

| | | Comparative | Comparative |
|---|---|---|---|
| | Example 17 | example 8 | example 9 |
| Tape adhering | ◯ | Δ | X |
| property | | | |
| Blocking resistance | ◯ | Δ | Δ |
| Bio-based content (%) | 30.2 | 29.2 | 21.7 |

From the results of table 11, it was apparent that the gravure ink of Example 17 had superior properties.

### (Preparation and Evaluation of an Adhesive Composition)

### <Preparation of Base Compound>

The components were mixed according to the composition shown in table 12 to prepare base compounds AD-1 to AD-4. The unit in table was gram (g).

### <Preparation of Curing Agent>

Isocyanate (Duranate TPA-100 manufactured by Asahi Kasei Corporation) 100 g and ethyl acetate 100 g were mixed to obtain a curing agent (H-1).

TABLE 12

| | AD-1 | AD-2 | AD-3 | AD-4 |
|---|---|---|---|---|
| Resin of synthesis example 1 | 100 | 100 | | |
| Resin of synthesis example 6 | | | 100 | |
| Resin of synthesis example 7 | | | | 100 |
| Ethyl acetate | 50 | 75 | 75 | 75 |
| Bisphenol A epoxy resin | | 25 | 25 | 25 |
| (manufactured by Tohto Kasei | | | | |
| Co., Ltd. YD-012) | | | | |

### Examples 18 and 19, Comparative Examples 10 and 11

The main compound and the curing agent were mixed in the proportion of 100:15 (weight ratio) according to the composition shown in the table 13 and the solid matter thereof was adjusted to be 30% by using ethyl acetate to obtain an adhesive solution.

### <Performance Test>

A multilayer film was prepared by adhering a polyester film to an aluminum foil using the adhesive solutions in examples and comparative examples and evaluated by the following performance test.

The adhesive composition was coated in such away that the coated amount was 4 to 5 g/m² by using a dry-laminating machine on a polyester film (LUMIRROR X-10S manufactured by TORAY INDUSTRIES, INC., film thickness 50 μm), the solvent was volatilized, and then an aluminum foil (thickness 50 μm) was laminated. The curing (aging) was conducted at 60° C. for 7 days to cure the adhesive composition. The obtained multilayer film was put in a glass bottle and the glass bottle was filled with water and sealed. The bottle was kept at 85° C. for 15 days or 30 days. The stored multilayer film was cut into pieces of 200 mm×15 mm and dried at room temperature for 6 hours. Then, T-type peeling test was conducted by using a tension testing machine at load velocity of 300 mm/min. according to ASTM D1876-61 method. The peeling strength between the polyester film and the aluminum foil of five test pieces were measured and the average value thereof was calculated.

The average value of the peeling strength was evaluated according to the following four categories.

A: The average value was 5N/15 mm or more and the laminate base material was broken (superior practice).
B: The average value was not less than 4N and less than 5N/15 mm and the peeling was occurred at the interface between the laminate base material and the adhesive composition (practicable).
C: The average value was not less than 2N and less than 4N/15 mm and the peeling was occurred at the interface between the laminate base material and the adhesive composition (barely practicable).
D: The average value was less than 2N/15 mm and the adhesive composition was agglutinate and broken.

The evaluation results were shown in table 13. The bio-based content was the value about the solid matter in the adhesive composition.

TABLE 13

| | Exam- | Exam- | Comparative | Comparative |
|---|---|---|---|---|
| | ple 18 | ple 19 | example 10 | example 11 |
| Base compound | AD-1 | AD-2 | AD-3 | AD-4 |
| Curing agent | H-1 | H-1 | H-1 | H-1 |
| Initial | A | A | B | C |
| After 15 days | A | A | B | D |
| After 30 days | B | A | C | D |
| Bio-based | 50.5 | 36.7 | 37.9 | 27.2 |
| content (%) | | | | |

### Synthesis Example 23

Trimethylolpropane 151.6 g, 1,3-propanediol 129.0 g, sebacic acid 457.1 g, and dibutyltin oxide 1.5 g were put in the same reaction vessel as that of synthesis example 1 and heated to 150° C. After the condensation reaction was conducted at ordinary pressure for 5 hours, it was heated to 220° C. The reaction vessel was connected with a vacuum pump, and the reaction was continued for four hours keeping the pressure inside the reaction vessel at 7 mmHg or less. It was confirmed that the acid value was 0.5 mgKOH/g or less.

### Synthesis Example 24

The synthesis was conducted by following the same decompression procedure as that of synthesis example 23 in the same composition as that of synthesis example 6 without the use of solvent.

### Synthesis Example 25

The synthesis was conducted by following the same decompression procedure as that of synthesis example 23 in the same composition as that of synthesis example 7 without the use of solvent.

### Preparation of Moisture-Curable Reactive Hot-Melt Adhesive Composition

### Example 20

Polyester polyol 310 g of synthesis example 23 was put in the same reaction vessel as that of synthesis example 23, mixed and dehydrated at 100° C. for 2 hours under reduced pressure. Then, JEFFCAT DMDEE (product name, manufactured by MITSUI FINE CHEMICALS, INC.) 0.15 g, and MILLIONATE MT (4,4′-MDI, manufactured by NIPPON POLYURETHANE INDUSTRY CO., LTD. product name) 355 g were put. The mixture was mixed at 100° C. for 2 hours in a nitrogen atmosphere and reacted to obtain a moisture-curable reactive hot-melt adhesive composition being solid at ordinary temperature (NCO/OH=2.7).

### Comparative Example 12

Polyester polyol 310 g of synthesis example 24 was put in the same reaction vessel as that of synthesis example 23, mixed and dehydrated at 100° C. for 2 hours under reduced pressure. Then, JEFFCAT DMDEE (product name, manufactured by MITSUI FINE CHEMICALS, INC.) 0.15 g, and MILLIONATE MT (4,4′-MDI, manufactured by NIPPON POLYURETHANE INDUSTRY CO., LTD. product name) 194 g were put. The mixture was mixed at 100° C. for 2 hours in a nitrogen atmosphere and reacted to obtain a moisture-curable reactive hot-melt adhesive composition being solid at ordinary temperature (NCO/OH=2.7).

### Comparative Example 13

Polyester polyol 310 g of synthesis example 25 was put in the same reaction vessel as that of synthesis example 23, mixed and dehydrated at 100° C. for 2 hours under reduced pressure. Then, JEFFCAT DMDEE (product name, manufactured by MITSUI FINE CHEMICALS, INC.) 0.15 g, and MILLIONATE MT (4,4′-MDI, manufactured by NIPPON POLYURETHANE INDUSTRY CO., LTD. product name) 106 g were put. The mixture was mixed at 100° C. for 2 hours in a nitrogen atmosphere and reacted to obtain a moisture-curable reactive hot-melt adhesive composition being solid at ordinary temperature (NCO/OH=2.7).

### <Test Evaluation Method>

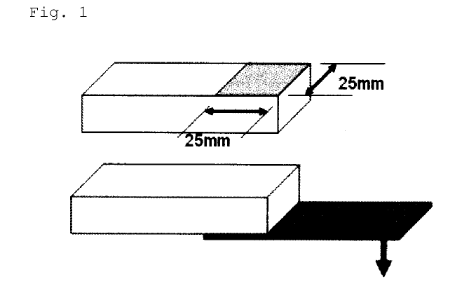

As shown in FIG. 1, the moisture-curable reactive hot-melt adhesive composition melted at 120° C. was applied on a particle board by using a 125 μm doctor blade from end to 25 mm length. Then, after a melamine decorative board was attached as shown in Fig., a test article was prepared by pressing at the linear pressure of 5 kg/cm. Right after the preparation, the test article was put at 30° C. atmosphere, and 2 minutes later the test article was set in such away that the attachment part of the melamine decorative board was hanged out of the test desk. Then, a weight 100 g was added at 12 mm from the end of the melamine decorative board, and a creep test was conducted toward 90° direction. The test article which supported the 100 g weight for 10 minutes or more was appraised as ◯. The test article which was peeled off in 10 minutes or less was appraised as x. The evaluation results were shown in table 14.

TABLE 14

| | | Comparative | Comparative |
|---|---|---|---|
| | Example 20 | example 12 | example 13 |
| Result of evaluation | ◯ | X | X |
| Bio-based content (%) | 37.0 | 34.6 | 25.6 |

### Synthesis of Polyfunctional Acrylate

### Synthesis Example 26

Polyester polyol 400 g of synthesis example 23 was put in the same reaction vessel as that of synthesis example 23, mixed and dehydrated at 100° C. under reduced pressure for 2 hours. Then, isophorone diisocyanate 165.4 g, hydroxyethyl acrylate 172.8 g, dibutyltin oxide 1.5 g, and methoxy hydroquinone 0.7 g were put and reacted at 80° C. for 4 hours and 120° C. for 2 hours.

### Synthesis Example 27

Polyester polyol 500.0 g of synthesis example 24 was put in the same reaction vessel as that of synthesis example 23, mixed and dehydrated at 100° C. under reduced pressure for 2 hours. Then, isophorone diisocyanate 113.2 g, hydroxyethyl acrylate 118.3 g, dibutyltin oxide 1.5 g, and methoxy hydroquinone 0.7 g were put and reacted at 80° C. for 4 hours and 120° C. for 2 hours.

### Synthesis Example 28

Polyester polyol 500.0 g of synthesis example 25 was put in the same reaction vessel as that of synthesis example 23, mixed and dehydrated at 100° C. under reduced pressure for 2 hours. Then, isophorone diisocyanate 62.0 g, hydroxyethyl acrylate 64.8 g, dibutyltin oxide 1.3 g, and methoxy hydroquinone 0.6 g were put and reacted at 80° C. for 4 hours and 120° C. for 2 hours.

### <Production of Ultraviolet Curable Adhesive Composition>

### Example 21, Comparative Examples 14 and 15

The ultraviolet curable adhesive compositions were prepared by mixing urethane acrylate oligomer, acrylate monomer, and a photo initiator being commercially available, according to the composition of example and comparative examples shown in table 15 until the mixture was homogeneously-mixed. The unit in table was gram (g).

Then, a PET film with 150 mm width×150 mm length×188 μm thickness was prepared and a release mask with 150 mm width×25 mm length×45 μm thickness was put on the PET film from the end, covering ⅙ area of the PET film. The ultraviolet curable resin composition having an arbitrary composition was spray coated completely over the both of the portion covered by the release mask and the portion uncovered by the release mask (being the portion which was uncovered by the release mask and the rest ⅚ area of the PET film). The ultraviolet curable resin composition with 150 mm width×125 mm length×100 μm thickness (applying the ultraviolet curable resin composition directly on the PET film with 100 μm thickness) on the portion uncovered by the release mask and the ultraviolet curable resin composition with 150 mm width×25 mm length×55 μm thickness (applying the ultraviolet curable resin composition with 55 μm thickness on the release mask with 45 μm thickness which was put on the PET film) on the portion covered by the release mask were formed and irradiated with ultraviolet light by using an ultraviolet irradiator (manufactured by USHIO INC., model number SP-7) 15 cm above for 5 seconds at 365 nm ultraviolet. After curing the ultraviolet curable resin composition, the release mask was removed and a specimen comprising the adhering portion of the ultraviolet curable resin composition to the PET film: 150 mm width×125 mm length×100 μm thickness and the portion of the ultraviolet curable resin composition not adhering to the PET film: 150 mm width×25 mm length×55 μm thickness was obtained. Further, the specimen was cut into six equal parts with 25 mm width, and specimens comprising the adhering portion of the ultraviolet curable resin composition to the PET film: 25 mm width×125 mm length×100 μm thickness and the portion of the ultraviolet curable resin composition not adhering to the PET film: 25 mm width×25 mm length×55 pm thickness were obtained. The adhering strength of the adhering portion of the ultraviolet curable resin composition to the PET film was examined by holding the portion of the ultraviolet curable resin composition not adhering to the PET film respectively by a clamp of the tension testing machine and conducting T-peel test at the crosshead speed of 50 mm/min. The adhering strength was measured 100 hours later at 85° C., and 100 hours later at 65° C. and 90% RH in addition to the initial state. The results were shown in table 15.

TABLE 15

| | | | | Compar. | Compar. |
|---|---|---|---|---|---|
| | | | Ex. 21 | Ex. 14 | Ex. 15 |
| Composi- | Polyfunc- | Synthesis | 70 | | |
| tion | tional | example 26 | | | |
| | acrylate | Synthesis | | 70 | |
| | | example 27 | | | |
| | | Synthesis | | | 70 |
| | | example 28 | | | |

| | | | | |
|---|---|---|---|---|
| | AMO | 30 | 30 | 30 |
| | Irgacure 184D | 1.5 | 1.5 | 1.5 |
| Adhering | Initial value | 590 | 90 | 60 |
| strength | 100 hours later at 85° C. | 490 | 80 | 50 |
| (N/m) | 100 hours later at 65° C. | 520 | 80 | 40 |
| | and 90% RH | | | |
| Bio-based | In the solid matter of | 40.7 | 39.3 | 36.2 |
| content (%) | the coating composition | | | |

AMO: acryloyl morpholine manufactured by KYOEISYA CHEMICAL Co., Ltd.
Irgacure 184D: 1-hydroxy-cyclohexyl-phenyl-ketone manufactured by BASF

From table 15, it was evidenced that the ultraviolet curable adhesive composition of example 21 had a superior adhering strength.

### Example 22 Production of Urethane Foam

Polyester resin 100 parts of synthesis example 1, water 1.2 parts, diethanolamine 1.5 parts, triethylene diamine 1.0 parts, L5309 (manufactured by Nippon Unicar Company Limited) 0.9 part, and CORONATE T80 (manufactured by NIPPON POLYURETHANE INDUSTRY CO., LTD.) 30 parts were put in a polypropylene vessel and mixed homogeneously using a mixer. Instantly, the mixture was poured into an aluminum mold with an opening of 100 mm×200 mm×200 mm at the top, and foamed to obtain urethane foam. The polyester resin to be used was the one obtained without adding butyl acetate after polymerization.

### Examples 23, 24 and Comparative Example 16

Urethane foams were produced by the same procedure as that of example 22 according to the composition shown in table 16.

TABLE 16

| | | | | Compar. |
|---|---|---|---|---|
| | Ex. 22 | Ex. 23 | Ex. 24 | Ex. 16 |
| Polyester resin of | 100 | | | |
| synthesis example 1 | | | | |
| Polyester resin of | | 100 | | |
| synthesis example 2 | | | | |
| Polyester resin of | | | 100 | |
| synthesis example 7 | | | | |
| Polyester resin of | | | | 100 |
| synthesis example 8 | | | | |
| Water | 1.2 | 1.2 | 1.2 | 1.2 |
| Diethanol amine | 1.5 | 1.5 | 1.5 | 1.5 |
| Triethylene diamine | 1.0 | 1.0 | 1.0 | 1.0 |
| Silicone foam stabilizer | 0.9 | 0.9 | 0.9 | 0.9 |
| L5309 (manufactured | | | | |
| by Nippon Unicar | | | | |
| Company Limited) | | | | |
| CORONATE T80 (manu- | 30 | 33 | 9 | 14 |
| factured by NIPPON | | | | |
| POLYURETHANE | | | | |
| INDUSTRY CO., LTD.) | | | | |
| Appearance | ◯ | ◯ | ◯ | X |
| Texture | ◯ | ⊚ | ◯ | X |
| Bio-based content | 43 | 60 | 37 | 85 |

The evaluations in table 16 were conducted according to the following criterions.

### (Appearance)

The polyurethane foam were observed by eye and evaluated according to the following criterion.

◯: There was no defect.
x: There were defects.

### (Texture)

The feeling was evaluated according to the following criterion when one pressed the obtained foam with a finger

⊚: Very good

### ◯: Good

### x: Bad

The polyurethane foams of examples were superior in appearance and texture, keeping a high bio-based content. The polyester resin used in comparative example 16 had the crystallinity, and there was a problem about the miscibility with an isocyanate, so the obtained urethane foam was inferior in appearance and texture.

### INDUSTRIAL APPLICABILITY

The polyester resin of the present disclosure can be used as a mixed component or synthesis material for a polyester resin, a coating composition, resin particles, cosmetics, a matte coating composition, an acrylic monomer, and an energy curable coating composition. The resin can be used as a mixed component for inks including a gravure ink, and adhesive compositions including an energy curable adhesive composition and a moisture-curable reactive hot-melt adhesive composition, and a material for a polyurethane foam.

1. A polyester resin obtained by polymerizing a monomer composition containing
10 to 90 weight % of a linear dicarboxylic acid and/or diol having at least 8 carbon atoms (I), 5 to 80 weight % of a branched dicarboxylic acid and/or diol having at least 4 carbon atoms (II-1) and/or 2 to 40 weight % of at least one polyfunctional monomer (II-2) selected from the group consisting of polyols, polycarboxylic acids and hydroxycarboxylic acids having 3 or more functional groups respectively and
which has the number average molecular weight of 500 to 5000 and is amorphous.

2. The polyester resin according to claim 1,
wherein part or all of the linear dicarboxylic acid and/or diol having at least 8 carbon atoms (I) is sebacic acid.

3. The polyester resin according to claim 1, obtained by polymerizing a monomer composition containing 0 to 88 weight % of other monomer (III).

4. The polyester resin according to claim 3,
wherein the other monomer (III) comprises at least one monomer selected from the group consisting of succinic acid, polyethylene glycol, 1,3-propanediol, and 1,4-butanediol.

5. A polyester resin which is obtained by polymerizing a monomer composition containing
a dicarboxylic acid monomer comprising at least one dicarboxylic acid (a) selected from the group of succinic acid and sebacic acid, a diol monomer comprising 1,4-butanediol and/or 1,3-propanediol (b), and at least one polyfunctional monomer (c) selected from the group of polyols, polycarboxylic acids and hydroxycarboxylic acids having 3 or more functional groups respectively,
wherein said monomer composition comprises 40 to 95 weight % of bio-based materials relative to the all resin materials,
the number average molecular weight (Mn) thereof is 500 to 5000, and
said polyester resin is amorphous.

6. A coating composition containing
a polyester resin (A) and a curing agent (B), wherein the polyester resin (A) is the polyester resin according to claim 1.

7. The coating composition according to claim 6,
which comprises a hydroxyl group-containing acrylic resin (C).

8. An adhesive composition containing a polyester resin (A) and a curing agent(B),
wherein said polyester resin (A) is the polyester resin according to claim 1.

9. A polyurethane foam obtained by foaming a composition containing the polyester resin (A) according to claim 1, and a polyisocyanate (B-1).

10. A resin particle obtained by suspension polymerization of the coating composition according to claim 6, and having the number average particle diameter of 2 to 20 μm.

11. A cosmetic containing the resin particle according to claim 10.

12. A matte coating composition containing the resin particle according to claim 10.

13. The matte coating composition according to claim 12, being a water-borne coating composition.

14. An acrylic monomer obtained by converting an end of the polyester resin according to claim 1 to an acryloyl group.

15. An energy curable coating composition of which part or all is the acrylic monomer according to claim 14.

16. A curable resin composition having a terminal isocyanate group obtained by reacting the polyester resin (A) according to claim 1, and a polyisocyanate (B-1).

17. A moisture-curable reactive hot-melt adhesive composition containing the curable resin composition according to claim 16.

18. A resin composition having a terminal hydroxyl group obtained by reacting the polyester resin (A) according to claim 1, and a polyisocyanate (B-1).

19. A print ink composition containing the resin composition according to claim 18.

20. An energy curable resin by reacting the polyester resin (A) according to claim 1, a compound having an unsaturated group and a functional group which is reactive with an isocyanate group, and a polyisocyanate (B-1).

21. An energy curable adhesive composition containing the energy curable resin according to claim 20.
